SEQUENCE LISTING (11 sequences)

SEQ ID NO: 1 - DNA, 454 bp, adenovirus serotype 5
  misc_feature (1)..(454): Nucleotides 1-454 of adenovirus serotype 5
catcatcaataatataccttattttggattgaagccaatatgataatgagggggtggagtttgtgacgtggcgcggggcgtgggaacggggcgggtgacgtagtagtgtgcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagc

SEQ ID NO: 2 - DNA, 2286 bp, adenovirus serotype 5
  misc_feature (1)..(2286): Nucleotides 3511 to 5796 of adenovirus serotype 5
tactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggt

SEQ ID NO: 3 - DNA, 2585 bp, adenovirus serotype 5
  misc_feature (1)..(2585): Nucleotides 3511 to 6095 of adenovirus serotype 5
tactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagta

SEQ ID NO: 4 - DNA, 1004 bp, adenovirus serotype 5
  misc_feature (1)..(1004): Nucleotides 34931 to 35934 of adenovirus serotype 5
gctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 5 - DNA, 35934 bp, adenovirus serotype 5
catcatcaataatataccttattttggattgaagccaatatgataatgagggggtggagtttgtgacgtggcgcggggcgtgggaacggggcgggtgacgtagtagtgtgcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 6 - DNA, 39 bp, Artificial Sequence
  Primer sequence for amplification of Influnza strain A/Panama/2007/99 HA gene
cacacaggtaccgccatgaagactatcattgctttgagc

SEQ ID NO: 7 - DNA, 32 bp, Artificial Sequence
  Primer sequence for amplification of Influnza strain A/Panama/2007/99 HA gene
cacacaggtacctcaaatgcaaatgttgcacc

SEQ ID NO: 8 - DNA, 37 bp, Artificial Sequence
  Primer sequence for amplification of Influenza strain B/Hong Kong/330/01 HA gene
cacacaggtaccgccatgaaggcaataattgtactac

SEQ ID NO: 9 - DNA, 41 bp, Artificial Sequence
  Primer sequence for amplification of Influenza strain B/Hong Kong/330/01 HA gene
cacacaggtaccagtagtaacaagagcatttttcaataacg

SEQ ID NO: 10 - DNA, 36 bp, Artificial Sequence
  Primer for amplification of tetracycline resistance gene from plasmid pBR322
gagctcggtaccttctcatgtttgacagcttatcat

SEQ ID NO: 11 - DNA, 36 bp, Artificial Sequence
  Primer for amplification of tetracycline resistance gene from plasmid pBR322
tctagaggtaccaacgctgcccgagatgcgccgcgt

(19) United States
(12) Patent Application Publication
(10) Pub. No.: US 2009/0175897 A1
(43) Pub. Date: Jul. 9, 2009

(54) SYSTEM FOR RAPID PRODUCTION OF HIGH-TITER AND REPLICATION-COMPETENT ADENOVIRUS-FREE RECOMBINANT ADENOVIRUS VECTORS

(76) Inventors: De-chu C. Tang, Birmingham, AL (US); Jianfeng Zhang, Birmingham, AL (US); Kent R. Van Kampen, Birmingham, AL (US)

Correspondence Address: FROMMER LAWRENCE & HAUG, 745 FIFTH AVENUE- 10TH FL., NEW YORK, NY, 10151, US

(21) Appl. No.: 11/943,901

(22) Filed: Nov. 21, 2007

Related U.S. Application Data
(63) Continuation-in-part of application No. PCT/US2006/020350, filed on May 23, 2006.
(60) Provisional application No. 60/683,638, filed on May 23, 2005.

(51) Int. Cl. A61K 39/12; C12N 15/63; C12N 15/74; A61K 35/76; C12N 15/87; C12N 1/20

(52) U.S. Cl. 424/199.1; 435/320.1; 435/477; 435/489; 424/93.2; 435/466; 435/252.8

(57) ABSTRACT

The present invention relates generally to the fields of gene therapy, immunology, and vaccine technology. More specifically, the invention relates to a novel system that can rapidly generate high titers of adenovirus vectors that are free of replication-competent adenovirus (RCA). Also provided are methods of generating these RCA-free adenoviral vectors, immunogenic or vaccine compositions comprising these RCA-free adenovirus vectors, methods of expressing a heterologous nucleic acid of interest in these adenovirus vectors and methods of eliciting immunogenic responses using these adenovirus vectors.

### CROSS-REFERENCE TO RELATED APPLICATIONS

This application is a continuation in-part of International Patent Application No. PCT/US2006/020350 filed May 23, 2006 and published as WO 2006/127956 on Nov. 30, 2006, which claims priority to U.S. Provisional Application Ser. No. 60/683,638 filed May 23, 2005.

Mention is also made of U.S. patent application Ser. Nos. 10/052,323, filed Jan. 18, 2002; 10/116,963, filed Apr. 5, 2002; 10/346,021, filed Jan. 16, 2003 and U.S. Pat. Nos. 6,706,693; 6,716,823; 6,348,450, and PCT/US/98/16739, filed Aug. 13, 1998.

Each of these applications, patents, and each document cited in this text, and each of the documents cited in each of these applications, patents, and documents (“application cited documents”), and each document referenced or cited in the application cited documents, either in the text or during the prosecution of the applications and patents thereof, as well as all arguments in support of patentability advanced during prosecution thereof, are hereby incorporated herein by reference.

### FIELD OF THE INVENTION

The present invention relates generally to the fields of immunology, gene therapy, and vaccine technology. More specifically, the invention relates to a novel system that can rapidly generate high titers of adenovirus vectors that are free of replication-competent adenovirus (RCA). Also provided are methods of generating these RCA-free adenoviral vectors, immunogenic or vaccine compositions comprising these RCA-free adenovirus vectors, methods of expressing a heterologous nucleic acid of interest in these adenovirus vectors and methods of eliciting immunogenic responses using these adenovirus vectors.

### BACKGROUND OF THE INVENTION

Influenza virus is a resurging, as well as emerging, microbial threat to public health. Infection of the respiratory tract by the virus is usually accompanied with coughing, fever and myalgia. The emergence of lethal influenza strains (Subbarao et al., 1998) and development of enabling technology to generate designer influenza viruses (Hoffmann et al., 2002; Neumann et al., 1999) has raised warning signs that dissemination of virulent influenza strains or man-made viruses encoding exogenous toxins by malicious human intent as a lethal weapon or incapacitating agent could cripple a region. The currently available, clinically licensed influenza vaccines consist of trivalent inactivated viruses that have been administered intramuscularly since the early 1940s (Pfleiderer et al., 2001). Annual fall vaccinations using these vaccines are effective in protecting people against this contagious disease (Nichol et al., 1995). However, the requirement for embryonated chicken eggs to produce the vaccine limits the speed of vaccine production. It is conceivable that a shortage of influenza vaccines will occur when new influenza virus strains emerge beyond calculation, chicken farms are crippled by avian influenza, and/or the production facility becomes contaminated, as in 2004.

More recently, a live attenuated influenza virus vaccine (FluMist™) has been developed as a needle-free alternative for influenza vaccination (Hilleman, 2002). The live attenuated vaccine is administered directly to the respiratory tract by intranasal sprays to prevent influenza in healthy children, adolescents and adults (ages 5-49 years). Like inactivated influenza virus vaccines, live attenuated influenza virus vaccines are also produced in embryonated chicken eggs. Although presence of chicken pathogens in eggs is not a problem for formaldehyde-killed virus vaccines, it is a biohazard for live attenuated influenza virus vaccines. Potentially harmful reassortments generated by recombination between live attenuated and wild influenza viruses present another biohazardous concern. Intranasal inoculation of live attenuated influenza vaccine is also associated with mild adverse events, such as runny nose, sore throat, or low-grade fever. Moreover, the live attenuated virus may destroy epithelial cells in the upper respiratory tract during replication, paving the way for secondary infections with pulmonary complications (Hilleman, 2002; Marwick, 2000).

The requirement to produce live attenuated and inactivated influenza virus vaccines in embryonated chicken eggs poses a major obstacle for streamlined manufacture of influenza vaccines because the process is time-consuming and some influenza virus strains do not propagate to high titers in eggs (Van Kampen et al., 2005). The demonstration that humans can be effectively and safely immunized by intranasal and topical application of adenovirus (Ad)-vectored influenza vaccines (Van Kampen et al., 2005) represents a new approach for the manufacture of influenza vaccines in a timely manner independent of embryonated chicken eggs.

Adenovirus is advantageous as a vaccine carrier because Ad vectors are capable of transducing both mitotic and postmitotic cells in situ (Shi et al., 1999), stocks containing high titers of virus (greater than 10¹¹ pfu per ml) can be prepared, making it possible to transduce cells in situ at high multiplicity of infection (MOI). Moreover, the Ad vectors are safe, based on its long-term use as a vaccine. The virus can induce high levels of heterologous nucleic acid expression, and the vector can be engineered to a great extent with versatility. Results have shown that the potency of an E1/E3-defective Ad5 vector as a nasal vaccine carrier is not suppressed by any preexisting immunity to Ad5 in animal models (Shi et al., 2001; Xiang et al., 1996). There is also no correlation between the potency of an Ad5-vectored nasal influenza vaccine and preexisting anti-Ad5 neutralizing antibody titer in humans (Van Kampen et al., 2005). Unlike gene therapy, Ad-vectored vaccines trigger an immune response through a cascade of immunologic reactions without the requirement for a critical level of heterologous nucleic acid expression. Replication-defective Ad-vectored nasal influenza vaccine should be safer than FluMist™ because the latter replicates in the respiratory tract and may contribute to the generation of new influenza virus strains through genetic reassortment with other circulating strains or recombinant forms. Moreover, manufacture of Ad-vectored influenza vaccine can be streamlined, as it does not require embryonated chicken eggs.

The conventional approach to construct a replication-defective recombinant Ad vector requires a series of time-consuming and labor-intensive steps involving homologous recombination between two transfected plasmids in mammalian packaging cells (Graham and Prevec, 1995). The finding that homologous recombination can be carried out in E. coli (Chartier et al., 1996; He et al., 1998) streamlined the procedure by allowing recombination to occur overnight in bacterial cells and obviating the need for plaque purification. The AdEasy system (He et al., 1998) exemplifies a fast-track system for generating recombinant Ad by homologous recombination in E. coli. See FIG. 6. Typically, a linearized shuttle vector plasmid encoding kanamycin (Kan) resistance is mixed with an adenoviral backbone plasmid (such as, for example, pAdEasy1) encoding ampicillin (Amp) resistance, followed by co-transformation into competent E. coli BJ5183 cells. Recombinants are subsequently selected for Kan resistance and identified by size, in conjunction with restriction endonuclease analysis. Finally, recombinant Ad vectors are generated by transfecting the recombinant plasmid into a mammalian packaging cell line (e.g., 293 cells).

A key step in producing a recombinant vector in E. coli in the AdEasy system can be enhanced by pre-selecting the Ad backbone plasmid prior to the delivery of the shuttle vector plasmid (Zeng et al., 2001). It is conceivable that only a small fraction of the pAdEasy1 plasmid pool may be allowed to persist in E. coli cells following transformation, because there is a high chance for a large plasmid [pAdEasy1 is 33 kb in size (He et al., 1998)] to be defective by, for example, the generation of nicks along its long DNA strands), and/or the efficiency for connecting a large plasmid to the cellular replication machinery may be low. Homologous recombination between a shuttle vector plasmid and an Ad backbone plasmid that is unable to exist as a replicon in E. coli cells is thus counterproductive for generating selectable recombinant plasmids, because such recombinants are abortive. The two-step AdEasier system (Zeng et al., 2001) ensures that homologous recombination occurs in a productive manner by eliminating defective and non-replicating Ad backbone plasmids in advance, thereby allowing a higher success rate during the selection for recombinants (AdEasy™ XL adenoviral vector system; Strategies 15(3): 58-59, 2002). Overall, this two-step transformation protocol may have broad utility in systems that involve homologous recombination in bacteria.

A critical issue for E1-deleted Ad vectors generated from human 293 cells is the emergence of replication-competent adenovirus (RCA). These contaminants arise through homologous recombination between identical sequences framing the E1 locus displayed by 293 cells, and the vector backbones (Robert et al., 2001; Zhu et al., 1999). RCA represents a biohazard because, like wild-type Ad, it can replicate in an infected host and potentially may cause disease. RCA-free Ad vectors have been generated in PER.C6 cells using PER.C6-compatible shuttle plasmids, such as pAdApt (Fallaux et al., 1998). Ad5 nucleotides 459-3510 in PER.C6 genome preclude double crossover-type homologous recombination with pAdApt-based shuttle plasmids (Crucell) that do not contain any overlapping sequences. Elimination of RCA in Ad stocks reduces the risk of exposure to the potential oncogene E1a and pathogenesis induced by replication of Ad in the host.

However, use of the PER.C6-amenable pAdApt-based shuttle plasmids is not amenable to homologous recombination in E. coli with pAdEasy1 because its “left arm” adenoviral sequence is missing. Generation of recombinant Ad vectors by co-transfecting pAdApt and an Ad backbone plasmid into PER.C6 cells (Fallaux et al., 1998) is time-consuming and labor-intensive. Typically, approximately 1-2 months of time can be saved for construction of a new Ad vector by using the AdEasy system with homologous recombination taking place in E. coli cells without 2-3 cycles of plaque purification.

Consequently, there is a need in the art to rapidly manufacture safe influenza vaccines, preferably using an adenoviral vector system. However, current adenoviral vectors, especially those generated from human cells such as 293 cells, can carry the risk of disease, primarily through the production of RCA. The present invention addresses both of these problems by providing a novel system for rapidly producing adenovirus-based vaccines or immunogenic compositions that also comprise the added benefit of increased safety.

### SUMMARY OF THE INVENTION

A rapid production system for generating influenza vaccines has long been sought to aid in the battle against annual influenza outbreaks. The emergence of lethal influenza strains (Subbarao et al., 1998) and the potential for designer influenza viruses to be used as bioweapons (Hoffmann et al., 2002; Neumann et al., 1999) underscores the urgency to develop new techniques for rapid production of influenza vaccines. The present invention addresses these problems in the art by providing, inter alia, a novel adenoviral vector and method for generating high-titer vaccines by generating RCA (replication-competent adenovirus)-free Ad vectors encoding heterologous nucleic acids, such as but not limited to, influenza antigens in a timely manner. The process eliminates the requirement for growing influenza viruses in embryonated chicken eggs (Van Kampen et al., 2005), expedites administration of non-replicating influenza vaccines by nasal spray (Shi et al., 2001; Van Kampen et al., 2005), and reduces production time as well as costs.

In a first aspect of the present invention, a recombinant adenoviral vector is provided, comprising a first adenoviral sequence comprising SEQ ID NO:1, a promoter sequence, a multiple cloning site (MCS), a transcriptional terminator, a second adenoviral sequence comprising SEQ ID NO:2, a third adenoviral sequence comprising SEQ ID NO:4, wherein SEQ ID NO:2 and SEQ ID NO:4 comprise overlapping sequences that allow homologous recombination to occur in a prokaryotic cell between the recombinant adenoviral shuttle plasmid and an adenoviral backbone plasmid.

In one embodiment, the promoter is selected from the group consisting of a cytomegalovirus (CMV) major immediate-early promoter, a simian virus 40 (SV40) promoter, a β-actin promoter, an albumin promoter, an Elongation Factor 1-α (EF1-α) promoter, a PγK promoter, a MFG promoter, a herpes virus promoter, a Rous sarcoma virus promoter, or any other eukaryotic promoters.

The transcriptional terminator can be the SV40 polyadenylation signal, or any other eukaryotic polyadenylation signals. The bacterial origin of replication can be derived from the pBR322 origin of replication. In another embodiment, the antibiotic resistance genes in adenoviral shuttle and backbone plasmids are selected from the group consisting of ampicillin resistance gene, kanamycin resistance gene, chloramphenicol resistance gene, tetracycline resistance gene, hygromycin resistance gene, bleomycin resistance gene, and zeocin resistance gene.

The prokaryotic cell can be E. coli, preferably E. coli BJ5183 cells.

In a preferred embodiment, the adenoviral shuttle vector is pAdHigh, comprising a first adenoviral sequence comprising sequences 1-454 derived from adenovirus serotype 5, a promoter sequence, a MCS, a transcriptional terminator, a second adenoviral sequence comprising sequences 3511 to 6055 derived from adenovirus serotype 5 containing the pIX promoter, a bacterial origin of replication, and an antibiotic resistance gene, wherein the first and second adenoviral sequences comprise sequences that allow homologous recombination to occur in a prokaryotic cell between the recombinant adenoviral shuttle plasmid and an adenoviral backbone plasmid.

Another aspect of the present invention provides a method of generating a recombinant adenovirus that is substantially free of replication-competent adenovirus (RCA), comprising co-transforming a first shuttle plasmid and a second shuttle plasmid into a prokaryotic cell, wherein the first shuttle plasmid comprises a first adenoviral sequence and a first antibiotic resistance gene; and wherein the second shuttle plasmid comprises a second adenoviral sequence that contains additional adenoviral sequences not present in the first shuttle plasmid, and a second antibiotic resistance gene that is different from the first antibiotic resistance gene, wherein the co-transforming allows homologous recombination to occur between the first and second shuttle plasmids and wherein the prokaryotic transformants expressing both of the antibiotic resistance genes in the first and second shuttle plasmids comprise a first recombined adenoviral shuttle plasmid; recovering the first recombined shuttle adenoviral plasmid (pAdHighβ) from the prokaryotic cell; co-transforming the AdHigh shuttle plasmid and an adenoviral backbone plasmid into prokaryotic cells (e.g., E. coli BJ5183), wherein the prokaryotic transformants produce a second recombined adenoviral plasmid encoding a transgene; recovering the second recombined adenoviral plasmid from the prokaryotic cell; transfecting PER.C6 cells with the second recombined adenoviral plasmid; and recovering the recombinant adenovirus from the PER.C6 cells, wherein the recombinant adenovirus is substantially free of RCA.

Other cells containing Ad5 sequences 459-3510 can also be used as the packaging cell line for producing RCA-free Ad vectors with the AdHigh system.

In one embodiment to generate the pAdHigh shuttle plasmid, the first shuttle plasmid is pShuttle-CMV. In another embodiment, the second shuttle plasmid is pAdApt (Havenga, M. J., et al, 2001; von der Thüsen, J. H. et al, 2004).

The additional adenoviral sequences present in pAdHigh but missing in pShuttleCMV (He et al., 1998) comprise adenoviral nucleotides 342 to 454 of adenovirus serotype 5 and adenoviral nucleotides 3511 to 3533 from adenovirus serotype 5. The segment between nucleotides 3511-3533 is part of the adenoviral pIX promoter. Lack of a functional pIX promoter may explain why the AdEasy system generates high titer of Ad in 293 cells but not in PER.C6 cells because the former expresses pIX whereas the latter does not.

The prokaryotic cell can be E. coli, preferably E. coli BJ5183.

Another aspect of the present invention provides a method of generating a recombinant adenovirus that is substantially free of replication-competent adenovirus (RCA), comprising digesting a first and second shuttle plasmid with one or more restriction endonucleases, wherein the first shuttle plasmid comprises a first adenoviral sequence and wherein the second shuttle plasmid comprises additional adenoviral sequences not present in the first shuttle plasmid; excising a fragment encompassing the additional adenoviral sequences from the second shuttle plasmid; inserting the fragment into appropriate sites to replace the counterpart fragment of the first shuttle plasmid, thereby resulting in a first recombined adenoviral plasmid with genetic defects (e.g., the defective pIX promoter) repaired; co-transforming the first recombined adenoviral shuttle plasmid (pAdHighα) and an adenoviral backbone plasmid into prokaryotic cells, wherein the prokaryotic transformants produce a second recombined adenoviral plasmid; recovering the second recombined adenoviral plasmid from the prokaryotic cell; transfecting the second recombined adenoviral plasmid into PER.C6 cells; and recovering the recombinant adenovirus from the cells, wherein the recombinant adenovirus is substantially free of RCA.

The invention also provides immunogenic compositions comprising a recombinant adenovirus that is substantially free of replication-competent adenovirus (RCA) expressing one or more heterologous nucleic acids of interest, in admixture with pharmaceutically acceptable excipients.

In one embodiment, the one or more heterologous nucleic acids of interest comprise an influenza gene derived from influenza strains comprising influenza A, influenza B, influenza C, circulating recombinant forms, hybrid forms, clinical isolates, and field isolates. The influenza gene can comprise influenza hemagglutinin gene, influenza matrix gene, influenza neuraminidase, and influenza nuclear protein gene. The immunogenic composition can further comprise an adjuvant.

Another aspect of the present invention provides immunogenic compositions comprising a recombinant adenovirus that is substantially free of replication-competent adenovirus (RCA) expressing one or more influenza immunogens, in admixture with pharmaceutically acceptable excipients.

In another aspect of the present invention, a method of expressing one or more heterologous nucleic acids of interest in a recombinant adenovirus that is substantially free of replication-competent adenovirus (RCA) is provided, comprising the steps of digesting an adenoviral vector DNA of the invention with one or more restriction endonucleases, thereby linearizing the adenoviral vector; ligating one or more heterologous nucleic acids into the adenoviral vector, wherein the one or more heterologous nucleic acids are operably linked to a promoter sequence; transfecting the adenoviral vector DNA into PER.C6 or other packaging cells; and recovering the recombinant adenovirus expressing the one or more heterologous nucleic acids of interest from the cell.

The invention also provides a method of eliciting an immunogenic response to influenza in a subject in need thereof, comprising administering an immunologically effective amount of the composition of the invention to the subject.

The invention further provides a method of introducing and expressing one or more heterologous nucleic acids in a cell of interest, comprising contacting the cell with a recombinant adenovirus that is substantially free of replication-competent adenovirus (RCA), wherein the recombinant adenovirus expresses the one or more heterologous nucleic acids, and culturing the cell or maintaining the animal harboring the cell under conditions sufficient to express the heterologous nucleic acids.

The invention also provides a kit comprising the pAdHigh shuttle plasmid of the invention, an adenoviral backbone plasmid, and E. coli BJ5183 cells.

These and other embodiments are disclosed or are obvious from and encompassed by, the following Detailed Description.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1 is a plasmid map of the pShuttleCMV shuttle plasmid.

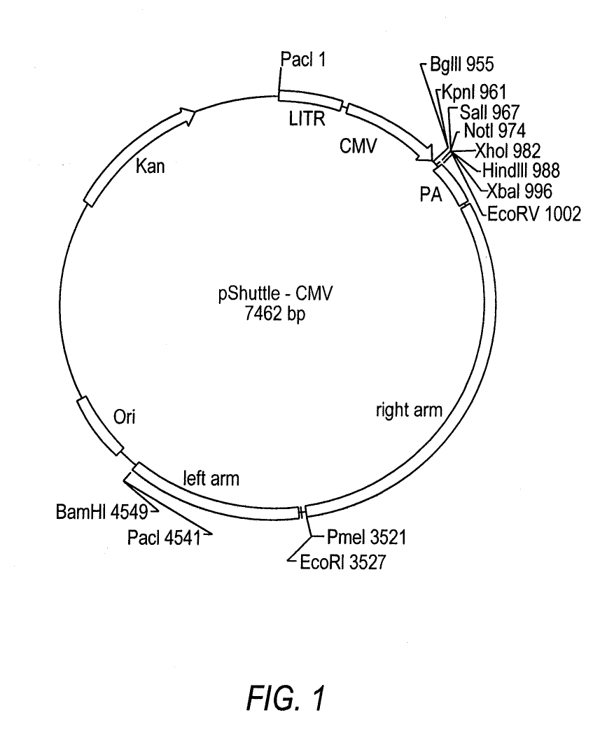

FIG. 2 is a plasmid map of the pAdApt shuttle plasmid.

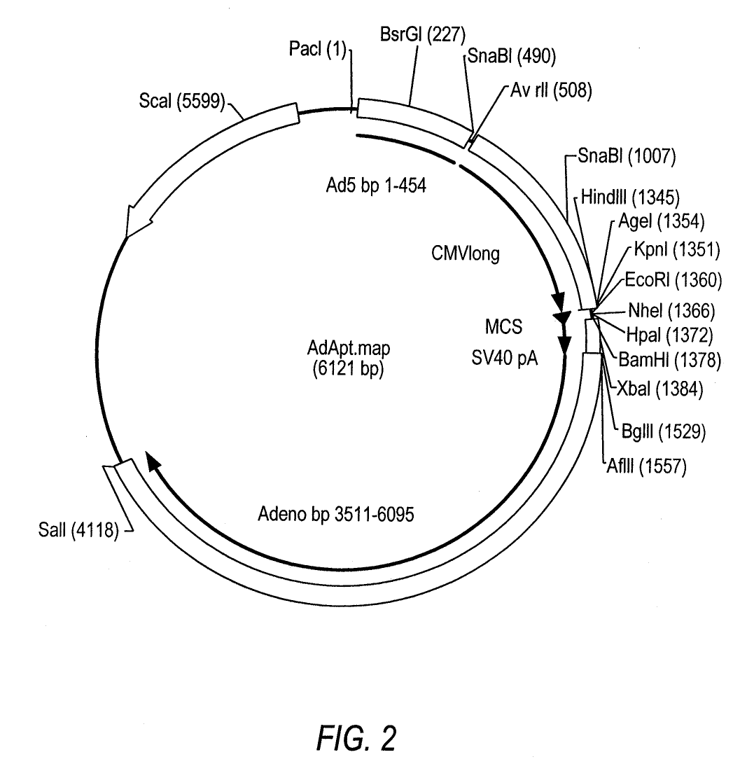

FIG. 3 depicts homologous recombination between pShuttleCMV shuttle plasmid and pAdApt shuttle plasmid. pShuttle-CMV encodes the kanamycin (Kan) resistance gene, pAdApt-Tc encodes both ampicillin (Amp) and tetracycline (Tc) resistance genes. Only recombinants can confer resistance to both Kan and Tc. Individual segments in plasmids are labeled by specific colors and are indicated by specific colored legends.

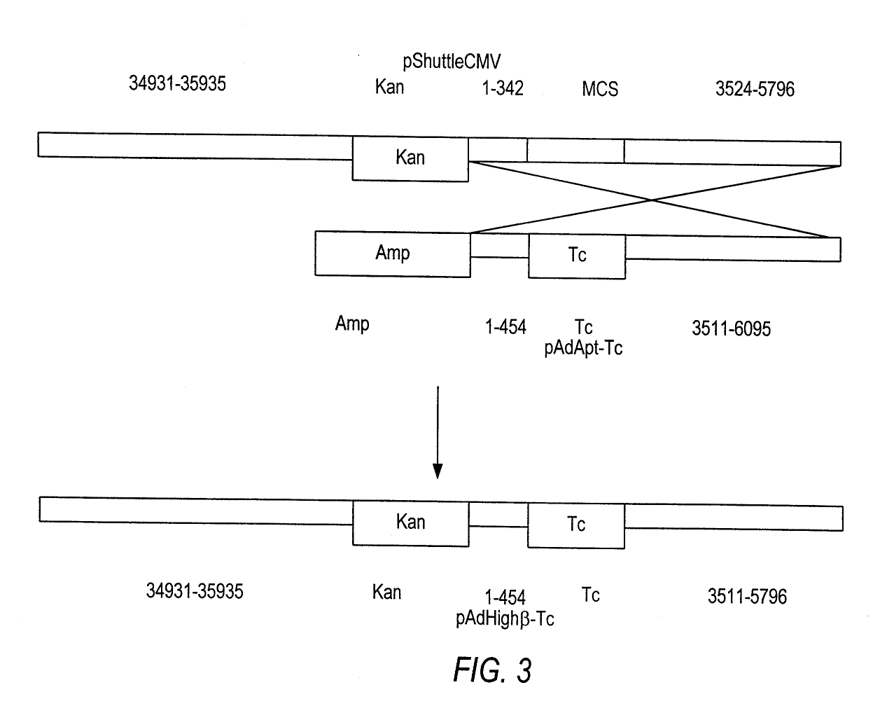

FIG. 4 is a plasmid map of the pAdHighβ shuttle plasmid.

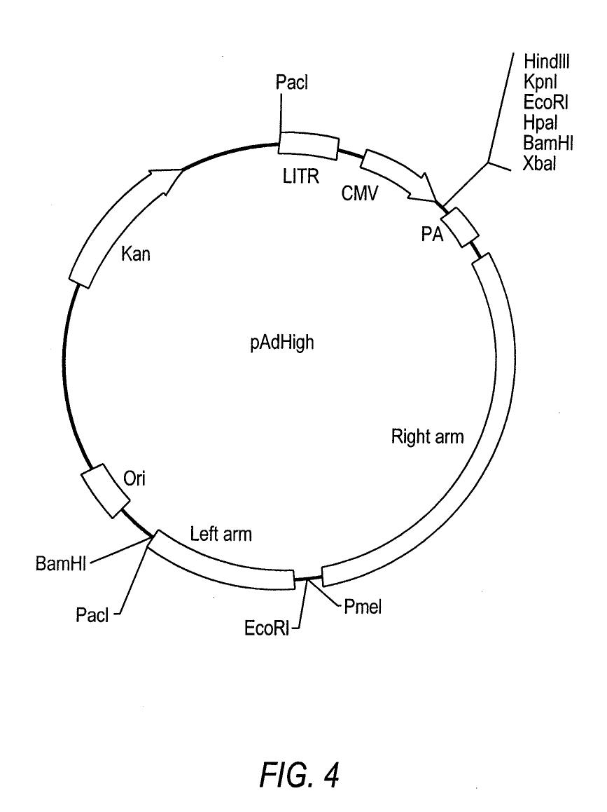

FIG. 5 is a general schematic depicting the construction of a recombinant Ad vector using pAdHigh and an Ad backbone plasmid.

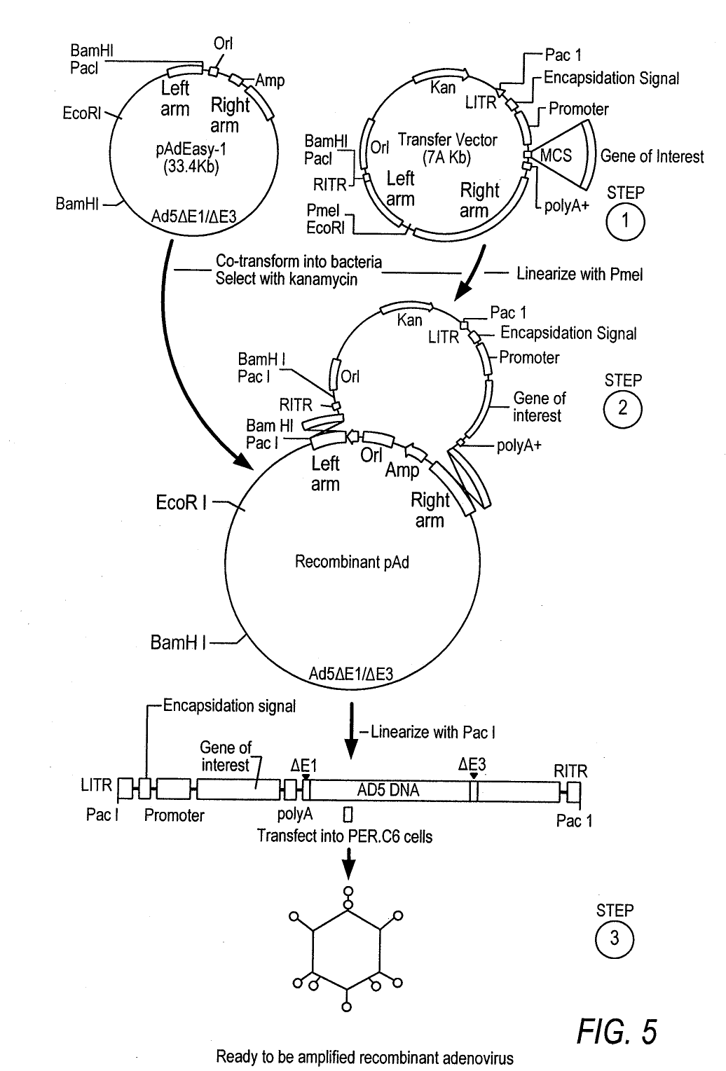

FIG. 6 is a graph showing the propagation of AdApt-, AdEasy-, and AdHighα-derived adenovirus vectors encoding an influenza HA gene in 293 and PER.C6 cells

FIG. 7 is a graph showing effectiveness of AdHighα- and AdApt-derived adenovirus vectors in eliciting hemagglutination-inhibition antibody titers.

SEQ ID NO: 1 refers to nucleotides 1 to 454 of adenovirus serotype 5.

SEQ ID NO: 2 refers to nucleotides 3511 to 5796 of adenovirus serotype 5.

SEQ ID NO: 3 refers to nucleotides 3511 to 6095 of adenovirus serotype 5.

SEQ ID NO: 4 refers to nucleotides 34931 to 35935 of adenovirus serotype 5.

### DETAILED DESCRIPTION OF THE INVENTION

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “nucleic acid” or “nucleic acid sequence” refers to a deoxyribonucleic or ribonucleic oligonucleotide in either single- or double-stranded form. The term encompasses nucleic acids, e.g., oligonucleotides, containing known analogues of natural nucleotides. The term also encompasses nucleic-acid-like structures with synthetic backbones, see e.g., Eckstein, 1991; Baserga et al., 1992; Milligan, 1993; WO 97/03211; WO 96/39154; Mata, 1997; Strauss-Soukup, 1997; Samstag, 1996.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the excision and ligation of nucleic acids having various coding regions or domains or promoter sequences from different sources into an expression cassette or vector for expression of, e.g., inducible or constitutive expression of polypeptide coding sequences in the vectors of invention.

The term “heterologous” when used with reference to a nucleic acid, indicates that the nucleic acid is in a cell or a virus where it is not normally found in nature; or, comprises two or more subsequences that are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences from unrelated genes arranged in a manner not found in nature; e.g., a human gene operably linked to a promoter sequence inserted into an adenovirus-based vector of the invention. As an example, a heterologous nucleic acid of interest can encode an immunogenic gene product, wherein the adenovirus is administered therapeutically or prophylactically as a vaccine or vaccine composition. Heterologous sequences can comprise various combinations of promoters and sequences, examples of which are described in detail herein.

An “antigen” is a substance that is recognized by the immune system and induces an immune response. A similar term used in this context is “immunogen”.

The term “inverted terminal repeat sequence” or “ITR” refers to the common usage of the term with respect to adenoviruses and includes all ITR sequences and variations thereof that are functionally equivalent, e.g., the term refers to sets of sequences (motifs) which flank (on the right and left) the linear adenovirus genome and are necessary for replication of the adenovirus genomic nucleic acid. The Ad sequences of the vectors and vector systems of the invention are flanked by ITRs, preferably derived from a serotype 5 adenovirus. There is a high degree of sequence conservation within the ITR between adenoviruses of different serotypes (see, e.g., Schmid, 1995).

A “subject” in the context of the present invention can be a vertebrate, such as a mammal, bird, reptile, amphibian or fish; more advantageously a human, or a companion or domesticated or food-producing or feed-producing or livestock or game or racing or sport animal such as, but not limited to, bovines, canines, felines, caprines, ovines, porcines, equines, and avians. Preferably, the vertebrate is a human. Since the immune systems of all vertebrates operate similarly, the applications described can be implemented in all vertebrate systems.

“Expression” of a gene or nucleic acid encompasses not only cellular gene expression, but also the transcription and translation of nucleic acid(s) in cloning systems and in any other context.

The term “gene product” refers primarily to proteins and polypeptides encoded by other nucleic acids (e.g., non-coding and regulatory RNAs such as tRNA, sRNPs).

As used herein, a “vector” is a tool that allows or facilitates the transfer of an entity from one environment to another. By way of example, some vectors used in recombinant DNA techniques allow entities, such as a segment of DNA (such as a heterologous DNA segment), to be transferred into a target cell. The present invention comprehends recombinant adenovirus vectors.

The term “plasmid” refers to a DNA transcription unit comprising a polynucleotide according to the invention and the elements required for its recombination, replication into Ad, and expression of transgenes in hosts. Preference is given to the circular plasmid form, which may or may not be supercoiled. The linear form also falls within the context of this invention.

With respect to exogenous DNA for expression in a vector (e.g., encoding an epitope of interest and/or an antigen and/or a therapeutic) and documents providing such exogenous DNA, as well as with respect to the expression of transcription and/or translation factors for enhancing expression of nucleic acid molecules, and as to terms such as “epitope of interest”, “therapeutic”, “immune response”, “immunological response”, “protective immune response”, “immunological composition”, “immunogenic composition”, and “vaccine composition”, inter alia, reference is made to U.S. Pat. No. 5,990,091 issued Nov. 23, 1999, and WO 98/00166 and WO 99/60164, and the documents cited therein and the documents of record in the prosecution of that patent and those PCT applications; all of which are incorporated herein by reference. Thus, U.S. Pat. No. 5,990,091 and WO 98/00166 and WO 99/60164 and documents cited therein and documents or record in the prosecution of that patent and those PCT applications, and other documents cited herein or otherwise incorporated herein by reference, can be consulted in the practice of this invention; and, all exogenous nucleic acid molecules, promoters, and vectors cited therein can be used in the practice of this invention. In this regard, mention is also made of U.S. Pat. Nos. 6,706,693; 6,716,823; 6,348,450; U.S. patent application Ser. Nos. 10/424,409; 10/052,323; 10/116,963; 10/346,021; and WO9908713, published Feb. 25, 1999, from PCT/US98/16739.

As used herein, the terms “immunogenic composition” and “immunological composition” and “immunogenic or immunological composition” cover any composition that elicits an immune response against the heterologous nucleic acids of interest expressed from the adenoviral vectors and viruses of the invention; for instance, after administration into a subject, elicits an immune response against the targeted immunogen or antigen of interest. The terms “vaccinal composition” and “vaccine” and “vaccine composition” covers any composition that induces a protective immune response against the antigen(s) of interest, or which efficaciously protects against the antigen; for instance, after administration or injection into the subject, elicits an protective immune response against the targeted antigen or immunogen or provides efficacious protection against the antigen or immunogen expressed from the inventive adenovirus vectors of the invention. The term “pharmaceutical composition” means any composition comprising a vector expressing a therapeutic protein as, for example, erythropoietin (EPO) or an immunomodulatory protein, such as, for example, GM-CSF.

An “immunologically effective amount” is an amount or concentration of the recombinant vector encoding the gene of interest, that, when administered to a subject, produces an immune response to the gene product of interest.

A “circulating recombinant form” refers to recombinant viruses that have undergone genetic reassortment among two or more subtypes or strains. Another term used in the context of the present invention is “hybrid form”.

“Clinical isolates” refer to, for example, frequently used laboratory strains of viruses that are isolated from infected patients and are reasserted in laboratory cells or subjects with laboratory-adapted master strains of high-growth shuttle viruses.

“Field isolates” refer to viruses that are isolated from infected patients or from the environment.

The methods of the invention can be appropriately applied to prevent diseases as prophylactic vaccination or provide relief against symptoms of disease as therapeutic vaccination.

The recombinant vectors of the present invention can be administered to a subject either alone or as part of an immunological composition. The recombinant vectors of the invention can also be used to deliver or administer a protein to a subject of interest by in vivo expression of the protein.

It is noted that immunological products and/or antibodies and/or expressed products obtained in accordance with this invention can be expressed in vitro and used in a manner in which such immunological and/or expressed products and/or antibodies are typically used, and that cells that express such immunological and/or expressed products and/or antibodies can be employed in in vitro and/or ex vivo applications, e.g., such uses and applications can include diagnostics, assays, ex vivo therapy (e.g., wherein cells that express the gene product and/or immunological response are expanded in vitro and reintroduced into the host or animal), etc., see U.S. Pat. No. 5,990,091, WO 99/60164 and WO 98/00166 and documents cited therein. Further, expressed antibodies or gene products that are isolated from herein methods, or that are isolated from cells expanded in vitro following herein administration methods, can be administered in compositions, akin to the administration of subunit epitopes or antigens or therapeutics or antibodies to induce immunity, stimulate a therapeutic response and/or stimulate passive immunity.

The term “adenovirus” as used herein is intended to encompass all adenoviruses, including the Atadenovirus, Mastadenovirus, and Aviadenovirus genera. To date, over fifty-one human serotypes of adenoviruses have been identified (see, e.g., Fields et al., Virology 2, Ch. 67 (3d ed., Lippincott-Raven Publishers). The adenovirus can be of serogroup A, B, C, D, E, or F. The adenovirus can be a serotype 2 (Ad2), serotype 11 (Ad11), serotype 35 (Ad35) or, preferably, serotype 5 (Ad5), but are not limited to these examples.

Adenovirus is a non-enveloped DNA virus. Vectors derived from adenoviruses have a number of features that make them particularly useful for gene transfer. As used herein, a “recombinant adenovirus vector” is an adenovirus vector that carries one or more heterologous nucleotide sequences (e.g., two, three, four, five or more heterologous nucleotide sequences). For example, the biology of the adenoviruses is characterized in detail, the adenovirus is not associated with severe human pathology, the virus is extremely efficient in introducing its DNA into the host cell, the virus can infect a wide variety of cells and has a broad host range, the virus can be produced in large quantities with relative ease, and the virus can be rendered replication detective by deletions in the early region 1 (“E I”) of the viral genome.

The genome of adenovirus (“Ad”) is a linear double-stranded DNA molecule of approximately 36,000 base pairs (“bp”) with a 55-kDa terminal protein covalently bound to the 5′ terminus of each strand. The Ad DNA contains identical Inverted Terminal Repeats (“ITRs”) of about 100 bp, with the exact length depending on the serotype. The viral origins of replication are located within the ITRs exactly at the genome ends. DNA synthesis occurs in two stages. First, the replication proceeds by strand displacement, generating a daughter duplex molecule and a parental displaced strand. The displaced strand is single stranded and can form a so called “panhandle” intermediate, which allows replication initiation and generation of a daughter duplex molecule. Alternatively, replication may proceed from both ends of the genome simultaneously, obviating the requirement to form the panhandle structure.

During the productive infection cycle, the viral genes are expressed in two phases: the early phase, which is the period up to viral DNA replication, and the late phase, which coincides with the initiation of viral DNA replication. During the early phase only the early gene products, encoded by regions E1, E2, E3 and E4, are expressed, which carry out a number of functions that prepare the cell for synthesis of viral structural proteins (Berk, A. J. 1986). During the late phase, the late viral gene products are expressed in addition to the early gene products and host cell DNA and protein synthesis are shut off. Consequently, the cell becomes dedicated to the production of viral DNA and of viral structural proteins (Tooze, J., 1981).

The E1 region of adenovirus is the first region of adenovirus expressed after infection of the target cell. This region consists of two transcriptional units, the E1A and E1B genes, both of which are required for oncogenic transformation of primary (embryonal) rodent cultures. The main functions of the E1A gene products are to induce quiescent cells to enter the cell cycle and resume cellular DNA synthesis, and to transcriptionally activate the E1B gene and the other early regions (E2, E3 and E4) of the viral genome. Transfection of primary cells with the E1A gene alone can induce unlimited proliferation (immortalization), but does not result in complete transformation. However, expression of E1A in most cases results in induction of programmed cell death (apoptosis), and only occasionally is immortalization obtained (Jochemsen et al., 1987). Co-expression of the E1B gene is required to prevent induction of apoptosis and for complete morphological transformation to occur. In established immortal cell lines, high-level expression of E1A can cause complete transformation in the absence of E1B (Roberts et al., 1981).

The E1B encoded proteins assist E1A in redirecting the cellular functions to allow viral replication. The E1B 55 kD and E4 33 kD proteins, which form a complex that is essentially localized in the nucleus, function in inhibiting the synthesis of host proteins and in facilitating the expression of viral genes. Their main influence is to establish selective transport of viral mRNAs from the nucleus to the cytoplasm, concomitantly with the onset of the late phase of infection. The E1B 21 kD protein is important for correct temporal control of the productive infection cycle, thereby preventing premature death of the host cell before the virus life cycle has been completed. Mutant viruses incapable of expressing the E1B 21 kD gene product exhibit a shortened infection cycle that is accompanied by excessive degradation of host cell chromosomal DNA (deg-phenotype) and in an enhanced cytopathic effect (cyt-phenotype; Telling et al., 1994). The deg and cyt phenotypes are suppressed when in addition the E1A gene is mutated, indicating that these phenotypes are a function of E1A (White et al., 1988). Furthermore, the E1B 21 kDa protein slows down the rate by which E1A switches on the other viral genes. It is not yet known by which mechanisms E1B 21 kD quenches these E1A dependent functions.

In contrast to, for example, retroviruses, adenoviruses do not integrate into the host cell's genome, are able to infect non-dividing cells, and are able to efficiently transfer recombinant genes in vivo (Brody et al., 1994). These features make adenoviruses attractive candidates for in vivo gene transfer of, for example, a heterologous nucleic acid of interest into cells, tissues or subjects in need thereof.

Embodiments of the invention employing adenovirus recombinants may include E1-defective or deleted, E3-defective or deleted, and/or E4-defective or deleted adenovirus vectors, or the “gutless” adenovirus vector in which all viral genes are deleted. The adenovirus vectors can comprise mutations in E1, E3, or E4 genes, or deletions in these or all adenoviral genes. The E1 mutation raises the safety margin of the vector because E1-defective adenovirus mutants are said to be replication-defective in non-permissive cells, and are, at the very least, highly attenuated. The E3 mutation enhances the immunogenicity of the antigen by disrupting the mechanism whereby adenovirus down-regulates MHC class I molecules. The E4 mutation reduces the immunogenicity of the adenovirus vector by suppressing the late gene expression, thus may allow repeated re-vaccination utilizing the same vector. The present invention comprehends adenovirus vectors of any serotype or serogroup that are deleted or mutated in E1, E3, E4, E1 and E3, and E1 and E4. The present invention also comprehends adenoviruses of the human Ad5 strain.

The “gutless” adenovirus vector is the latest model in the adenovirus vector family. Its replication requires a helper virus and a special human 293 cell line expressing both E1a and Cre, a condition that does not exist in natural environment; the vector is deprived of all viral genes, thus the vector as a vaccine carrier is non-immunogenic and may be inoculated multiple times for re-vaccination. The “gutless” adenovirus vector also contains 36 kb space for accommodating heterologous nucleic acid(s) of interest, thus allowing co-delivery of a large number of antigen or immunogens into cells.

Other adenovirus vector systems known in the art include the AdEasy system (He et al., 1998) and the subsequently modified AdEasier system (Zeng et al., 2001), which were developed to generate recombinant Ad vectors in 293 cells rapidly by allowing homologous recombination between shuttle plasmids and Ad backbone plasmids to occur in Escherichia coli cells overnight. However, a low level of RCA, which presents a potential biohazard for human application, contaminates Ad vectors produced in 293 cells. The creation of RCA is due to overlapping sequences between the Ad vector and 293 cell genome (Fallaux et al., 1998; Zhu et al., 1999).

Although RCA-free Ad vectors have been generated in PER.C6 cells after transfecting an Ad backbone plasmid in conjunction with a PER.C6-compatible shuttle plasmid that does not contain any overlapping sequences with the PER.C6 genome (Fallaux et al., 1998), the process for constructing Ad vectors through homologous recombination in human cell background is time-consuming when compared to the AdEasy recombination system in E. coli cells. AdEasy-derived Ad vectors can be generated in PER.C6 cells rapidly, however, this method yields a low titer [<10⁸ plaque-forming units (pfu) per ml], presumably due to defective sequences in pShuttleCMV (He et al., 1998) that are not complemented in trans by the PER.C6 packaging cell line.

Rapid and high-titer production of RCA-free Ad-vectored influenza vaccines can be achieved by repairing the defective sequences in pShuttleCMV to generate a new shuttle plasmid defined in an embodiment of the present invention, named pAdHigh. It is expected that an Ad-vectored influenza vaccine can be generated from AdHigh as rapidly as AdEasy because shuttle plasmids in both systems contain identical components for homologous recombination with the adenoviral backbone plasmid pAdEasy1 (He et al., 1998) in E. coli background, preferably E. coli BJ5183.

pAdEasy1 comprises adenoviral sequences that, when recombined with a shuttle plasmid such as pShuttle-CMV and pAdHigh expressing heterologous nucleic acids of interest, results in generation of an EL/E3-defective adenoviral genome encoding the heterologous nucleic acids (e.g., immunogens and/or therapeutic genes) packaged into an adenoviral capsid. The sequence of pAdEasy1 is well known in the art and is publicly and commercially available through Stratagene. In contrast to AdEasy-derived Ad vector, the AdHigh-derived Ad vector propagates to titers as high as that of a PER.C6-compatible vector-derived counterpart, and avoids RCA contamination when produced in PER.C6 cells because Ad sequences in AdHigh-derived Ad vectors are identical to their counterparts generated from the PER.C6-compatible shuttle plasmid pAdApt (Crucell; Leiden, Netherlands).

The present invention provides methods of generating a novel adenovirus shuttle plasmid that is amenable to production of RCA-free Ad vectors, comprising co-transforming a first and second shuttle plasmids into a prokaryotic cell, wherein the first shuttle plasmid comprises a subfragment of adenoviral sequence and a first antibiotic resistance gene, and wherein the second shuttle plasmid comprises a subfragment of adenoviral sequence that contains adenoviral sequences not present in the first shuttle plasmid, and a second antibiotic resistance gene that is different from the first antibiotic resistance gene. In this method, pAdHigh is generated by homologous recombination of two shuttle plasmids that comprise adenoviral sequences that are necessary for generating RCA-free recombinant adenoviruses.

The first shuttle plasmid can be pShuttleCMV, or another shuttle plasmid that comprises an adenoviral sequence useful in homologous recombination with adenoviral sequences derived from another plasmid. pShuttleCMV is commercially available and its sequence is in the public domain (He et al, 1998). pShuttleCMV comprises a multiple cloning site that is used to insert one or more heterologous nucleic acids of interest, which is operably linked to a CMV promoter. pShuttleCMV also comprises a kanamycin resistance gene.

A second shuttle plasmid, comprising a subfragment of adenoviral sequence containing additional adenoviral sequences not present in the first shuttle plasmid, can be pAdApt (Fallaux et al., 1998; von der Thüsen, J. H. et al, 2004; Havenga, M. J., 2001). The additional sequences present in the second shuttle plasmid such as pAdApt include sequences derived from Ad5, but can also comprise sequences from other adenovirus serotypes. These sequences comprise SEQ ID NO: 1, which corresponds to adenoviral sequences 1 to 454 from Ad5, and SEQ ID NO: 3, which corresponds to adenoviral sequences 3511 to 6095 from Ad5. The invention also comprehends the use of the corresponding sequences from other adenovirus serotypes, including but not limited to Ad2, Ad7, Ad11, and Ad35. The skilled artisan is familiar with methods of sequence alignment, such as BLAST (Altschul, S. F. et al, (1990), which can identify the appropriate sequences in other adenoviral serotypes or serogroups. Any shuttle plasmid that comprises these sequences, or sequence variants thereof, can be used in the methods of the invention.

The present invention concerns generating recombined adenoviral plasmids by homologous recombination of the first and second shuttle plasmids as described above, or by excising the additional adenoviral sequences from the second shuttle plasmid and inserting the sequences by ligation into the first shuttle plasmid. In one embodiment, the invention provides a method of generating pAdHigh, by digesting a first and second shuttle plasmid with one or more restriction endonucleases, wherein the first shuttle plasmid comprises a first adenoviral sequence and a first antibiotic resistance gene and wherein the second shuttle plasmid comprises additional adenoviral sequences not present in the first shuttle plasmid, inserting the additional adenoviral sequences into the first shuttle plasmid, thereby resulting in a first recombined adenoviral shuttle plasmid pAdHighα. One of skill in the art is familiar with methods of nucleic acid cloning and manipulation, without undue experimentation.

Recovery of plasmids is well-known in the art and can be achieved by lysis of prokaryotic transformants (such methods include, but are not limited to, French press, alkaline lysis, nitrogen cavitation) and purification of plasmids by cesium chloride centrifugation, ethanol precipitation, column chromatography (e.g., Qiagen prep), among others. Any method of transfecting cells can be used in the methods of the invention. Such methods include use of calcium phosphate precipitates, cationic lipids, liposomes, microinjection, and infection by viral delivery.

The adenovirus vectors of the present invention are useful for the delivery of nucleic acids to cells both in vitro and in vivo. In particular, the inventive vectors can be advantageously employed to deliver or transfer nucleic acids to animal, more preferably mammalian cells. Nucleic acids of interest include nucleic acids encoding peptides and proteins, preferably therapeutic (e.g., for medical or veterinary uses) or immunogenic (e.g., for vaccines) peptides or proteins.

Preferably, the codons encoding the heterologous nucleic acids of interest are “humanized” codons, e.g., the codons are those that appear frequently in highly expressed human genes instead of those codons that are frequently used by, for example, influenza. Such codon usage provides for efficient expression of the heterologous nucleic acid in human or other animal cells. Codon usage patterns are known in the literature for highly expressed genes of many species (e.g., Nakamura et al., 1996; Wang et al, 1998; McEwan et al. 1998).

As a further alternative, the adenovirus vectors can be used to infect a cell in culture or animals to express a desired gene product, e.g., to produce a protein or peptide of interest. Preferably, the protein or peptide is secreted into the medium and can be purified therefrom using routine techniques known in the art. Signal peptide sequences that direct extracellular secretion of proteins are known in the art and nucleotide sequences encoding the same can be operably linked to the nucleotide sequence encoding the peptide or protein of interest by routine techniques known in the art. Alternatively, the cells can be lysed and the expressed recombinant protein can be purified from the cell lysate. The cells may be eukaryotic. Preferably, the cell is an animal cell (e.g., insect, avian or mammalian), more preferably a mammalian cell. Also preferred are cells that are competent for transduction by adenoviruses.

Such cells include PER.C6 cells, 911 cells, and HEK293 cells. PER.C6 cells are useful, due to the ability of PER.C6 cells to propagate RCA-free Ad vectors. PER.C6 cells are primary human retinoblast cells transduced with an E1 gene segment that complements the production of replication-incompetent adenovirus, but is designed to prevent generation of RCA by homologous recombination. PER.C6 is described in WO 97/00326, published on Jan. 3, 1997, the contents of which are incorporated herein by reference. Additionally, it should be noted that HEK 293 cells (Graham et al, 1977) carry overlapping sequences that could recombine with the adenoviral sequences of the invention to produce RCA.

The present invention also provides vectors useful as vaccines. The immunogen or antigen can be presented in the adenovirus capsid, alternatively, the antigen can be expressed from a heterologous nucleic acid introduced into a recombinant adenovirus genome and carried by the inventive adenoviruses. The adenovirus vector can provide any immunogen of interest. Immunogens of interest are well-known in the art and include, but are not limited to, immunogens from human immunodeficiency virus (e.g., envelope proteins, such as gp160, gp120, gp41), influenza virus, gag proteins, cancer antigens, HBV surface antigen (to immunize against hepatitis), rabies glycoproteins, and the like. Additional examples of immunogens are detailed herein.

The heterologous nucleotide sequence(s) are preferably operably associated with the appropriate expression control sequences. Expression vectors include expression control sequences, such as an origin of replication (which can be bacterial origins, e.g., derived from bacterial vectors such as pBR322, or eukaryotic origins, e.g., autonomously replicating sequences (ARS)), a promoter, an enhancer, and necessary information processing sites, such as ribosome binding sites, RNA splice sites, polyadenylation sites, packaging signals, and transcriptional terminator sequences.

For example, the recombinant adenovirus vectors of the invention preferably contain appropriate transcription/translation control signals and polyadenylation signals (e.g., polyadenylation signals derived from bovine growth hormone, SV40 polyadenylation signal) operably associated with the heterologous nucleic acid sequence(s) to be delivered to the target cell. A variety of promoter/enhancer elements may be used depending on the level and tissue-specific expression desired. The promoter can be constitutive or inducible (e.g., the metallothionein promoter), depending on the pattern of expression desired. The promoter may be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) or tissue(s) of interest. Brain-specific, hepatic-specific, and muscle-specific (including skeletal, cardiac, smooth, and/or diaphragm-specific) promoters are contemplated by the present invention. Mammalian promoters are also preferred.

The promoter can advantageously be an “early” promoter. An “early” promoter is known in the art and is defined as a promoter that drives expression of a gene that is rapidly and transiently expressed in the absence of de novo protein synthesis. The promoter can also be a “strong” or “weak” promoter. The terms “strong promoter” and “weak promoter” are known in the art and can be defined by the relative frequency of transcription initiation (times per minute) at the promoter. A “strong” or “weak” promoter can also be defined by its affinity to poxyiral RNA polymerase.

More preferably, the heterologous nucleotide sequence(s) are operatively associated with, for example, a human cytomegalovirus (CMV) major immediate-early promoter, a simian virus 40 (SV40) promoter, a β-actin promoter, an albumin promoter, an Elongation Factor 1-α (EF1-α) promoter, a PγK promoter, a MFG promoter, or a Rous sarcoma virus promoter. Other expression control sequences include promoters derived from immunoglobin genes, adenovirus, bovine papilloma virus, herpes virus, and so forth. Any mammalian viral promoter can also be used in the practice of the invention. It has been speculated that driving heterologous nucleotide transcription with the CMV promoter results in down-regulation of expression in immunocompetent animals (see, e.g., Guo et al., 1996). Accordingly, it is also preferred to operably associate the heterologous nucleotide sequences with a modified CMV promoter that does not result in this down-regulation of heterologous nucleic acid expression.

The vectors of the invention can also comprise a multiple cloning site (“MCS”), which can advantageously be located downstream of the first promoter. The MCS provides a site for insertion of the heterologous nucleic acid molecules that are “in-frame” with the promoter sequence, resulting in “operably linking” the promoter sequence to the heterologous nucleic acid of interest. Multiple cloning sites are well known to those skilled in the art. As used herein, the term “operably linked” means that the components described are in a relationship permitting them to function in their intended manner.

Depending on the vector, selectable markers encoding antibiotic resistance may be present when used for in vitro amplification and purification of the recombinant vector, and to monitor homologous recombination between the shuttle plasmid and the adenoviral vector. The methods of the invention describe facilitating homologous recombination between a shuttle plasmid and an adenoviral vector at overlapping sequences. Each vector comprises a different antibiotic resistance gene, and by dual selection, recombinants expressing the recombined vector can be selected. Examples of such antibiotic resistance genes that can be incorporated into the vectors of the invention include, but are not limited to, ampicillin, tetracycline, neomycin, zeocin, kanamycin, bleomycin, hygromycin, chloramphenicol, among others.

In embodiments wherein there is more than one heterologous nucleotide sequence, the heterologous nucleotide sequences may be operatively associated with a single upstream promoter and one or more downstream internal ribosome entry site (IRES) sequences (e.g., the picornavirus EMC IRES sequence).

In embodiments of the invention in which the heterologous nucleotide sequence(s) will be transcribed and then translated in the target cells, specific initiation signals are generally required for efficient translation of inserted protein coding sequences. These exogenous translational control sequences, which may include the ATG initiation codon and adjacent sequences, can be of a variety of origins, both natural and synthetic.

Therapeutic peptides and proteins include, but are not limited to, cystic fibrosis transmembrane regulator protein (CFTR), dystrophin (including the protein product of dystrophin mini-genes, see, e.g, Vincent et al., 1993), utrophin (Tinsley et al., 1996), clotting factors (e.g., Factor XII, Factor IX, Factor X, etc.), erythropoietin, the LDL receptor, lipoprotein lipase, ornithine transcarbamylase, β-globin, α-globin, spectrin, α-antitrypsin, adenosine deaminase, hypoxanthine guanine phosphoribosyl transferase, β-glucocerebrosidase, sphingomyelinase, lysosomal hexosaminidase, branched-chain keto acid dehydrogenase, hormones, growth factors, cytokines, suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), and any other peptide or protein that has a therapeutic effect in a subject in need thereof.

Recombinant vectors provided by the invention can also code for immunomodulatory molecules, which can act as an adjuvant to provoke a humoral and/or cellular immune response. Such molecules include cytokines, co-stimulatory molecules, or any molecules that may change the course of an immune response. The molecule(s) can comprise genes that encode such immunomodulatory molecules such as, but not limited to, a GM-CSF gene, a B7-1 gene, a B7-2 gene, an interleukin-2 gene, an interleukin-12 gene and interferon genes. One of skill in the art can conceive of ways in which this technology can be modified to enhance still further the immunogenicity of antigens and/or immunogens.

The invention also relates to such methods wherein the exogenous nucleic acid molecule encodes one or more of an antigen or portion thereof, e.g., one or more of an epitope of interest from a pathogen, e.g., an epitope, antigen or gene product which modifies allergic response, an epitope antigen or gene product which modifies physiological function, influenza hemagglutinin, influenza nuclear protein, influenza M2, tetanus toxin C-fragment, anthrax protective antigen, anthrax lethal factor, rabies glycoprotein, HBV surface antigen, HIV gp120, HIV gp160, human carcinoembryonic antigen, malaria CSP, malaria SSP, malaria MSP, malaria pfg, and mycobacterium tuberculosis HSP; and/or a therapeutic or an immunomodulatory gene, a co-stimulatory gene and/or a cytokine gene.

According to a preferred embodiment of the present invention, the recombinant vectors express a nucleic acid molecule encoding or expressing influenza immunogens or antigens. In particular, any or all genes or open reading frames (ORFs) of influenza encoding the products can be isolated, characterized and inserted into vector recombinants. Preferred influenza genes or ORFs include, but are not limited to, hemagglutinin, nuclear protein, matrix, and neuraminidase. The resulting recombinant adenovirus vector is used to immunize or inoculate a subject.

The present invention also provides methods of eliciting an immune response to influenza. Influenza is an enveloped, single-stranded, negative-sense RNA virus that causes serious respiratory ailments throughout the world. It is the only member of the Orthomyxoviridae family and has been subgrouped into three types, A, B and C. Influenza virions consist of an internal ribonucleoprotein core (a helical nucleoprotein) containing the single-stranded RNA genome, and an outer lipoprotein envelope lined inside by a matrix protein (M). The segmented genome of influenza A consists of eight molecules (seven for influenza C) of linear, negative polarity, single-stranded RNAs which encode ten polypeptides, including: the RNA-directed RNA polymerase proteins (PB2, PB1 and PA) and nuclear protein (NP) which form the nucleocapsid; the matrix proteins (M1, M2); two surface glycoproteins which project from the lipoprotein envelope: hemagglutinin (HA) and neuraminidase (NA); and nonstructural proteins whose function is unknown (NS1 and NS2). Transcription and replication of the genome takes place in the nucleus and assembly occurs via budding on the plasma membrane. The viral genes can reassort (e.g., undergo homologous recombination) during mixed infections.

Influenza virus adsorbs via HA to sialyloligosaccharides in cell membrane glycoproteins and glycolipids. Following endocytosis of the virion, a conformational change in the HA molecule occurs within the cellular endosome which facilitates membrane fusion, thus triggering uncoating. The nucleocapsid migrates to the nucleus where viral mRNA is transcribed as the essential initial event in infection. Viral mRNA is transcribed by a unique mechanism in which viral endonuclease cleaves the capped 5′-terminus from cellular heterologous mRNAs which then serve as primers for transcription of viral RNA templates by the viral transcriptase. Transcripts terminate at sites 15 to 22 bases from the ends of their templates, where oligo(U) sequences act as signals for the template-independent addition of poly(A) tracts. Of the eight viral mRNA molecules so produced, six are monocistronic messages that are translated directly into the proteins representing HA, NA, NP and the viral polymerase proteins, PB2, PB1 and PA. The other two transcripts undergo splicing, each yielding two mRNAs, which are translated in different reading frames to produce M1, M2, NS1 and NS2. In other words, the eight viral mRNAs code for ten proteins: eight structural and two non-structural.

The Influenza A genome contains eight segments of single-stranded RNA of negative polarity, coding for nine structural and one nonstructural proteins. The nonstructural protein NS1 is abundant in influenza virus infected cells, but has not been detected in virions. NS1 is a phosphoprotein found in the nucleus early during infection and also in the cytoplasm at later times of the viral cycle (Krug et al., 1975). Studies with temperature-sensitive (ts) influenza mutants carrying lesions in the NS gene suggested that the NS1 protein is a transcriptional and post-transcriptional regulator of mechanisms by which the virus is able to inhibit host cell gene expression and to stimulate viral protein synthesis. Like many other proteins that regulate post-transcriptional processes, the NS1 protein interacts with specific RNA sequences and structures. The NS1 protein has been reported to bind to different RNA species including: vRNA, poly-A, U6 (sn)RNA, 5′ untranslated region as of viral mRNAs and ds RNA (Qiu et al., 1995; Qiu et al., 1994). Expression of the NS1 protein from cDNA in transfected cells has been associated with several effects: inhibition of nucleo-cytoplasmic transport of mRNA, inhibition of pre-mRNA splicing, inhibition of host mRNA polyadenylation and stimulation of translation of viral mRNA (Fortes et al., 1994; Enami, K. et al, 1994; de la Luna et al., 1995; Lu, Y. et al., 1994; Park et al., 1995).

Influenza A viruses possess a genome of eight single-stranded negative-sense viral RNAs (vRNAs) that encode a total of ten proteins. The influenza virus life cycle begins with binding of the HA to sialic acid-containing receptors on the surface of the host cell, followed by receptor-mediated endocytosis. The low pH in late endosomes triggers a conformational shift in the HA, thereby exposing the N-terminus of the HA2 subunit (the so-called fusion peptide). The fusion peptide initiates the fusion of the viral and endosomal membrane, and the matrix protein (M1) and RNP complexes are released into the cytoplasm. RNPs consist of the nuclear protein (NP), which encapsidates vRNA, and the viral polymerase complex, which is formed by the PA, PB 1, and PB2 proteins. RNPs are transported into the nucleus, where transcription and replication take place. The RNA polymerase complex catalyzes three different reactions: synthesis of an mRNA with a 5′ cap and 3′ polyA structure, of a full-length complementary RNA (cRNA), and of genomic vRNA using the cDNA as a template. Newly synthesized vRNAs, NP, and polymerase proteins are then assembled into RNPs, exported from the nucleus, and transported to the plasma membrane, where budding of progeny virus particles occurs. The neuramimidase (NA) protein plays a crucial role late in infection by removing sialic acid from sialyloligosaccharides, thus releasing newly assembled virions from the cell surface and preventing the self aggregation of virus particles. Although virus assembly involves protein-protein and protein-vRNA interactions, the nature of these interactions is largely unknown.

Although influenza B and C viruses are structurally and functionally similar to influenza A virus, there are some differences. For example, influenza B virus does not have a M2 protein with ion channel activity. Instead, the NB protein, a product of the NA gene, likely has ion channel activity and thus a similar function to the influenza A virus M2 protein. Similarly, influenza C virus does not have a M2 protein with ion channel activity. However, the CM1 protein is likely to have this activity.

Such influenza A strains include, but are not limited to, subtypes H10N4, H10N5, H10N7, H10N8, H10N9, H11N1, H11N13, H11N2, H11N4, H11N6, H11N8, H11N9, H12N1, H12N4, H12N5, H12N8, H13N2, H13N3, H13N6, H13N7, H14N5, H14N6, H15N8, H15N9, H16N3, H1N1, H1N2, H1N3, H1N6, H1N9, H2N1, H2N2, H2N3, H2N5, H2N7, H2N8, H2N9, H3N1, H3N2, H3N3, H3N4, H3N5, H3N6, H3N8, H4N1, H4N2, H4N3, H4N4, H4N5, H4N6, H4N8, H4N9, H5N1, H5N2, H5N3, H5N7, H5N8, H5N9, H6N1, H6N2, H6N4, H6N5, H6N6, H6N7, H6N8, H6N9, H7N1, H7N2, H7N3, H7N5, H7N7, H7N8, H8N4, H8N5, H9N1, H9N2, H9N3, H9N5, H9N6, H9N7, H9N8, H9N9, hybrid subtypes, circulating recombinant forms, clinical and field isolates. Their sequences are available from GenBank and viral stock may be available from the American Type Culture Collection, Rockville, Md. or are otherwise publicly available.

Influenza B strains include, but are not limited to, strains originating from Aichi, Akita, Alaska, Ann Arbor, Argentina, Bangkok, Beijing, Belgium, Bonn, Brazil, Buenos Aires, Canada, Chaco, Chiba, Chongqing, CNIC, Cordoba, Czechoslovakia, Daeku, Durban, Finland, Fujian, Fukuoka, Genoa, Guangdong, Guangzhou, Hannover, Harbin, Hawaii, Hebei, Henan, Hiroshima, Hong Kong, Houston, Hunan, Ibaraki, India, Israel, Johannesburg, Kagoshima, Kanagawa, Kansas, Khazkov, Kobe, Kouchi, Lazio, Lee, Leningrad, Lissabon, Los Angeles, Lusaka, Lyon, Malaysia, Maputo, Mar del Plata, Maryland, Memphis, Michigan, Mie, Milano, Minsk, Nagasaki, Nagoya, Nanchang, Nashville, Nebraska, The Netherlands, New York, NIB, Ningxia, Norway, Oman, Oregon, Osaka, Oslo, Panama, Paris, Parma, Perugia, Philippines, Pusan, Quebec, Rochester, Roma, Saga, Seoul, Shangdong, Shanghai, Shenzhen, Shiga, Shizuoka, Sichuan, Siena, Singapore, South Carolina, South Dakota, Spain, Stockholm, Switzerland, Taiwan, Texas, Tokushima, Tokyo, Trento, Trieste, United Kingdom, Ushuaia, USSR, Utah, Victoria, Vienna, Wuhan, Xuanwu, Yamagata, Yamanashi, Yunnan, hybrid subtypes, circulating recombinant forms, clinical and field isolates. Their sequences are available from GenBank and viral stock may be available from the American Type Culture Collection, Rockville, Md. or are otherwise publicly available.

Influenza C strains include, but are not limited to, strains originating from Aichi, Ann Arbor, Aomori, Beijing, Berlin, California, England, Great Lakes, Greece, Hiroshima, Hyogo, JHB, Johannesburg, Kanagawa, Kansas, Kyoto, Mississippi, Miyagi, Nara, New Jersey, Saitama, Sapporo, Shizuoka, Taylor, Yamagata, hybrid subtypes, circulating recombinant forms, clinical and field isolates. Their sequences are available from GenBank and viral stock may be available from the American Type Culture Collection, Rockville, Md. or are otherwise publicly available.

A preferred embodiment of the invention provides an immunogenic composition that comprises at least one, preferably three or more, influenza immunogens, such as hemagglutinin, that are derived from different geographical regions or which target different strains, circulating recombinant forms, clinical, or field isolates for a particular year. The current commercially available influenza vaccine is a trivalent vaccine comprising influenza hemagglutinin immunogens from the three most prevalent influenza strains or circulating recombinant forms, as determined by the World Health Organization. Such a vaccine can be made using the methods disclosed herein and is contemplated as part of the present invention. Also contemplated are immunogenic compositions comprising at least three different neuraminidase or nuclear protein influenza immunogens derived from strains or circulating recombinant forms of interest, which may originate in a specific geographical region.

Expression in the subject of the heterologous sequence, e.g. influenza immunogens, can result in an immune response in the subject to the expression products of the heterologous sequence. Thus, the recombinant vectors of the present invention may be used in an immunological composition or vaccine to provide a means to induce an immune response, which may, but need not be, protective. The molecular biology techniques used in the context of the invention are described by Sambrook et al. (1989).

Even further alternatively or additionally, in the immunogenic or immunological compositions encompassed by the present invention, the nucleotide sequence encoding the antigens can have deleted therefrom a portion encoding a transmembrane domain. Yet even further alternatively or additionally, the vector or immunogenic composition can further contain and express in a host cell a nucleotide sequence encoding a heterologous tPA signal sequence such as human tPA and/or a stabilizing intron, such as intron II of the rabbit β-globin gene.

The present invention also provides a method of delivering and/or administering a heterologous nucleotide sequence into a cell in vitro or in vivo. According to this method a cell is infected with at least one deleted adenovirus vector according to the present invention (as described in detail herein). The cell may be infected with the adenovirus vector by the natural process of viral transduction. Alternatively, the vector may be introduced into the cell by any other method known in the art. For example, the cell may be contacted with a targeted adenovirus vector (as described below) and taken up by an alternate mechanism, e.g., by receptor-mediated endocytosis. As another example the vector may be targeted to an internalizing cell-surface protein using an antibody or other binding protein.

The cell to be administered the inventive virus vectors can be of any type, including but not limited to neuronal cells (including cells of the peripheral and central nervous systems), retinal cells, epithelial cells (including dermal, gut, respiratory, bladder and breast tissue epithelium), muscle cells (including cardiac, smooth muscle, skeletal muscle, and diaphragm muscle), pancreatic cells (including islet cells), hepatic cells (e.g., parenchyma), fibroblasts, endothelial cells, germ cells, lung cells (including bronchial cells and alveolar cells), prostate cells, and the like. Moreover, the cells can be from any species of origin, as indicated above. Preferred are cells that are naturally transduced by adenoviruses. Examples of such cells that are transduced by adenoviruses include, but are not limited to, HEK 293 cells, PER.C6 cells, and 911 cells. In one embodiment, PER.C6 cells are used.

Reference is made to U.S. Pat. No. 6,716,823 issued Apr. 6, 2004; U.S. Pat. No. 6,706,693 issued Mar. 16, 2004; U.S. Pat. No. 6,348,450 issued Feb. 19, 2002; U.S. application Ser. Nos. 10/052,323 and 10,116,963; and 10/346,021, the contents of which are expressly incorporated herein by reference.

Reference is also made to U.S. Pat. No. 5,990,091 issued Nov. 23, 1999, Einat et al. or Quark Biotech, Inc., WO 99/60164, published Nov. 25, 1999 from PCT/US99/11066, filed May 14, 1999, Fischer or Rhone Merieux, Inc., WO98/00166, published Jan. 8, 1998 from PCT/US97/11486, filed Jun. 30, 1997 (claiming priority from U.S. application Ser. Nos. 08/675,556 and 08/675,566), van Ginkel et al., 1997, and Osterhaus et al., 1992), for information concerning expressed gene products, antibodies and uses thereof, vectors for in vivo and in vitro expression of exogenous nucleic acid molecules, promoters for driving expression or for operatively linking to nucleic acid molecules to be expressed, method and documents for producing such vectors, compositions comprising such vectors or nucleic acid molecules or antibodies, dosages, and modes and/or routes of administration (including compositions for nasal administration), inter alia, which can be employed in the practice of this invention; and thus, U.S. Pat. No. 5,990,091 issued Nov. 23, 1999, Einat et al. or Quark Biotech, Inc., WO 99/60164, published Nov. 25, 1999 from PCT/US99/11066, filed May 14, 1999, Fischer or Rhone Merieux, Inc., WO98/00166, published Jan. 8, 1998 from PCT/US97/11486, filed Jun. 30, 1997 (claiming priority from U.S. application Ser. Nos. 08/675,556 and 08/675,566), van Ginkel et al., 1997, and Osterhaus et al., 1992) and all documents cited or referenced therein and all documents cited or referenced in documents cited in each of U.S. Pat. No. 5,990,091 issued Nov. 23, 1999, Einat et al. or Quark Biotech, Inc., WO 99/60164, published Nov. 25, 1999 from PCT/US99/11066, filed May 14, 1999, Fischer or Rhone Merieux, Inc., WO98/00166, published Jan. 8, 1998 from PCT/US97/11486, filed Jun. 30, 1997 (claiming priority from U.S. application Ser. Nos. 08/675,556 and 08/675,566), van Ginkel et al., 1997, and Osterhaus et al., 1992) are hereby incorporated herein by reference. Information in U.S. Pat. No. 5,990,091 issued Nov. 23, 1999, WO 99/60164, WO98/00166, van Ginkel et al., 1997, and Osterhaus et al., 1992 can be relied upon for the practice of this invention (e.g., expressed products, antibodies and uses thereof, vectors for in vivo and in vitro expression of exogenous nucleic acid molecules, exogenous nucleic acid molecules encoding epitopes of interest or antigens or therapeutics and the like, promoters, compositions comprising such vectors or nucleic acid molecules or expressed products or antibodies, dosages, inter alia).

A vector can be administered to a patient or host in an amount to achieve the amounts stated for gene product (e.g., epitope, antigen, therapeutic, and/or antibody) compositions. Of course, the invention envisages dosages below and above those exemplified herein, and for any composition to be administered to an animal or human, including the components thereof, and for any particular method of administration, it is preferred to determine therefor: toxicity, such as by determining the lethal dose 50 (LD₅₀) in a suitable animal model e.g., rodent such as mouse; and, the dosage of the composition(s), concentration of components therein and timing of administering the composition(s), which elicit a suitable response, such as by titrations of sera and analysis thereof, e.g., by ELISA and/or seroneutralization analysis. Such determinations do not require undue experimentation from the knowledge of the skilled artisan, this disclosure and the documents cited herein.

Examples of compositions of the invention include liquid preparations for orifice, or mucosal, e.g., oral, nasal, anal, vaginal, peroral, intragastric, etc., administration such as suspensions, solutions, sprays, syrups or elixirs; and, preparations for parenteral, epicutaneous, subcutaneous, intradermal, intramuscular, intranasal, or intravenous administration (e.g., injectable administration) such as sterile suspensions or emulsions. Reference is made to U.S. Pat. No. 6,716,823 issued Apr. 6, 2004; U.S. Pat. No. 6,706,693 issued Mar. 16, 2004; U.S. Pat. No. 6,348,450 issued Feb. 19, 2002; U.S. application Ser. Nos. 10/052,323 and 10,116,963; and 10/346,021, the contents of which are incorporated herein by reference and which disclose immunization and delivery of immunogenic or vaccine compositions through a non-invasive mode of delivery, e.g. epicutaneous and intranasal administration.

The invention also comprehends sequential administration of inventive compositions or sequential performance of herein methods, e.g., periodic administration of inventive compositions such as in the course of therapy or treatment for a condition and/or booster administration of immunological compositions and/or in prime-boost regimens; and, the time and manner for sequential administrations can be ascertained without undue experimentation.

Further, the invention comprehends compositions and methods for making and using vectors, including methods for producing gene products and/or immunological products and/or antibodies in vivo and/or in vitro and/or ex vivo (e.g., the latter two being, for instance, after isolation therefrom from cells from a host that has had a non-invasive administration according to the invention, e.g., after optional expansion of such cells), and uses for such gene and/or immunological products and/or antibodies, including in diagnostics, assays, therapies, treatments, and the like.

Vector compositions are formulated by admixing the vector with a suitable carrier or diluent; and, gene product and/or immunological product and/or antibody compositions are likewise formulated by admixing the gene and/or immunological product and/or antibody with a suitable carrier or diluent; see, e.g., U.S. Pat. No. 5,990,091, WO 99/60164, WO 98/00166, documents cited therein, and other documents cited herein, and other teachings herein (for instance, with respect to carriers, diluents and the like).

In such compositions, the recombinant vectors may be in admixture with a suitable carrier, diluent, or excipient such as sterile water, physiological saline, glucose or the like. The compositions can also be lyophilized. The compositions can contain auxiliary substances, such as wetting or emulsifying agents, pH buffering agents, adjuvants, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

The quantity of recombinant vector to be administered will vary for the patient (host) and condition being treated and will vary from one or a few to a few hundred or thousand micrograms, e.g., 1 μg to 1 mg, from about 100 ng/kg of body weight to 100 mg/kg of body weight per day and preferably will be from 10 pg/kg to 10 mg/kg per day. When administering a recombinant adenovirus, an immunologically, therapeutically, or prophylactically effective dose can comprise 1×10⁷ to 1×10¹² viral particles or plaque-forming units (PFU). A vector can be non-invasively administered to a patient or host in an amount to achieve the amounts stated for gene product (e.g., epitope, antigen, therapeutic, and/or antibody) compositions. Of course, the invention envisages dosages below and above those exemplified herein, and for any composition to be administered to a subject, including the components thereof, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response, such as by titrations of sera and analysis thereof, e.g., by ELISA and/or seroneutralization analysis. Such determinations do not require undue experimentation from the knowledge of the skilled artisan, this disclosure and the documents cited herein.

Recombinant vectors can be administered in a suitable amount to obtain in vivo expression corresponding to the dosages described herein and/or in herein cited documents. For instance, suitable ranges for viral suspensions can be determined empirically. If more than one gene product is expressed by more than one recombinant, each recombinant can be administered in these amounts; or, each recombinant can be administered such that there is, in combination, a sum of recombinants comprising these amounts.

However, the dosage of the composition(s), concentration of components therein and timing of administering the composition(s), which elicit a suitable immunological response, can be determined by methods such as by antibody titrations of sera, e.g., by ELISA and/or seroneutralization assay analysis. Such determinations do not require undue experimentation from the knowledge of the skilled artisan, this disclosure and the documents cited herein. And, the time for sequential administrations can be likewise ascertained with methods ascertainable from this disclosure, and the knowledge in the art, without undue experimentation.

The immunogenic or immunological compositions contemplated by the invention can also contain an adjuvant. Suitable adjuvants include fMLP (N-formyl-methionyl-leucyl-phenylalanine; U.S. Pat. No. 6,017,537) and/or acrylic acid or methacrylic acid polymer and/or a copolymer of maleic anhydride and of alkenyl derivative. The acrylic acid or methacrylic acid polymers can be cross-linked, e.g., with polyalkenyl ethers of sugars or of polyalcohols. These compounds are known under the term “carbomer” (Pharmeuropa, Vol. 8, No. 2, June 1996). A person skilled in the art may also refer to U.S. Pat. No. 2,909,462 (incorporated by reference), which discusses such acrylic polymers cross-linked with a polyhydroxylated compound containing at least 3 hydroxyl groups: in one embodiment, a polyhydroxylated compound contains not more than 8 hydroxyl groups; in another embodiment, the hydrogen atoms of at least 3 hydroxyls are replaced with unsaturated aliphatic radicals containing at least 2 carbon atoms; in other embodiments, radicals contain from about 2 to about 4 carbon atoms, e.g., vinyls, allyls and other ethylenically unsaturated groups. The unsaturated radicals can themselves contain other substituents, such as methyl. The products sold under the name Carbopol® (Noveon Inc., Ohio, USA) are particularly suitable for use as an adjuvant. They are cross-linked with an allyl sucrose or with allylpentaerythritol, as to which, mention is made of the products Carbopol® 974P, 934P, and 971P.

As to the copolymers of maleic anhydride and of alkenyl derivative, mention is made of the EMA® products (Monsanto), which are copolymers of maleic anhydride and of ethylene, which may be linear or cross-linked, for example cross-linked with divinyl ether. Also, reference may be made to U.S. Pat. No. 6,713,068 and Regelson, W. et al., 1960; (incorporated by reference).

Cationic lipids containing a quaternary ammonium salt are described in U.S. Pat. No. 6,713,068, the contents of which are incorporated by reference, can also be used in the methods and compositions of the present invention. Among these cationic lipids, preference is given to DMRIE (N-(2-hydroxyethyl)-N,N-dimethyl-2,3-bis(tetradecyloxy)-1-propane ammonium; WO96/34109), advantageously associated with a neutral lipid, advantageously DOPE (dioleoyl-phosphatidyl-ethanol amine; Behr J. P. et al, 1994), to form DMRIE-DOPE.

A recombinant vaccine or immunogenic or immunological composition can also be formulated in the form of an oil-in-water emulsion. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is available under the name Provax® (IDEC Pharmaceuticals, San Diego, Calif.).

The recombinant adenovirus, or recombinant adenoviral vector expressing one or more heterologous nucleic acids of interest, e.g., vector according to this disclosure, can be preserved and/or conserved and stored either in liquid form, at about 5° C., or in lyophilized or freeze-dried form, in the presence of a stabilizer. Freeze-drying can be according to well-known standard freeze-drying procedures. The pharmaceutically acceptable stabilizers may be SPGA (sucrose phosphate glutamate albumin; Bovarnik et al., 1950), carbohydrates (e.g., sorbitol, mannitol, lactose, sucrose, glucose, dextran, trehalose), sodium glutamate (Tsvetkov, T. et al., 1983; Israeli, E. et al., 1993), proteins such as peptone, albumin or casein, protein containing agents such as skimmed milk (Mills, C. K. et al., 1988; Wolff, E. et al., 1990), and buffers (e.g., phosphate buffer, alkaline metal phosphate buffer). An adjuvant and/or a vehicle or excipient may be used to make soluble the freeze-dried preparations.

The invention will now be further described by way of the following non-limiting Examples, given by way of illustration of various embodiments of the invention and are not meant to limit the present invention in any fashion.

### EXAMPLES

### Example 1

### Production of a Replication-Defective Adenovirus Expressing Influenza HA

Two adenovirus (Ad) vectors encoding influenza HA were constructed using the AdEasy system. Two current influenza virus strains, [A/Panama/2007/99 (H₃N₂) and B/Hong Kong/330/01] that were selected for vaccine production in 2003-2004, were provided by The Centers for Disease Control and Prevention (CDC). The A/Panama/2007/99 HA gene was cloned by reverse transcription of the influenza RNA, followed by amplification of the HA gene with polymerase chain reaction (PCR) using the following primers:

TABLE 1
Primer Sequences for Amplification of Influenza Genes

| | |
|---|---|
| Strain | Primer Sequence |

| | |
|---|---|
| A/Panama/2007/99 | 5′-CACACAGGTACCGCCATGAAGACTATCATTGCTTTGAGC-3′ |
| | 5′-CACACAGGTACCTCAAATGCAAATGTTGCACC-3′ |
| B/Hong Kong/330/01 | 5′-CACACAGGTACCGCCATGAAGGCAATAATTGTACTAC-3′ |
| | 5′-CACACAGGTACCAGTAGTAACAAGAGCATTTTTCAATAACG-3′ |

These primers contain sequences that anneal to the 5′ and 3′ ends of the A/Panama/2007/99 HA gene, an eukaryotic ribosomal binding site (Kozak, 1986) immediately upstream from the HA initiation ATG codon, and KpnI sites for subsequent cloning. The KpnI fragment containing the full-length HA gene was inserted into the KpnI site of pShuttleCMV (He et al., 1998) in the correct orientation under transcriptional control of the cytomegalovirus (CMV) early promoter. An EL1/E3-defective Ad vector encoding the A/Panama/2007/99 HA (AdPNM2007/99.H3) was generated in human 293 cells using the AdEasy system. An Ad vector encoding the B/Hong Kong/330/01 HA gene (AdHK330/01.B) was constructed likewise using the primer sequences in Table 1.

Both Ad vectors were validated by DNA sequencing and propagated to a titer of 10¹¹ pfu/ml in 293 cells. Hemagglutination-inhibition (HI) antibodies against A/Panama/2007/99 and B/Hong Kong/330/01 were elicited in mice after intranasal instillation of AdPNM2007/99.H3- and AdHK330/01.B-vectored influenza vaccines, respectively. However, a low titer of Ad vectors (<10⁸ pfu per ml) were produced for both vectors when the recombinant plasmids generated in E. coli BJ5183 cells were transfected into PER.C6 cells instead of 293 cells. The PER.C6-generated AdPNM2007/99.H3 vector was sent to Molecular Medicine BioServices, Inc. (La Jolla, Calif.) for mass production in PER.C6 cells, and the titer was 2×10⁷ pfu/ml after 4 rounds of expansion. Production of Ad vectors to a low titer is not an inherent problem in PER.C6 cells because the present inventors (unpublished results) and others (Fallaux et al., 1998; Murakami et al., 2002) have shown that Ad vectors generated by pAdApt-based shuttle plasmids grow to high titers (>10¹¹ pfu/ml) in this cell line. The AdEasy system does not appear to be compatible with PER.C6 cells and cannot be utilized for high-titer production of RCA-free Ad vectors.

Although construction of Ad-vectored influenza vaccines is faster than the conventional egg-dependent production system, even in the absence of the AdEasy system, the AdEasy system or an equivalent can be further accelerated by allowing homologous recombination between shuttle plasmids and Ad backbone plasmids to occur in E. coli cells overnight. Overall, one to two months of time can be saved if the AdEasy system is used to construct new Ad vectors instead of the conventional method for Ad construction. This timesaving procedure is meaningful for production of influenza vaccines, because a new influenza virus strain may become pandemic within this timeframe.

However, the generation of RCA in 293 cells and the incompatibility between the AdEasy system and the PER.C6 cell line are obstacles that prevent rapid and high-titer production of Ad vectors without RCA contamination. It is conceivable that the low-titer production of AdEasy-derived Ad vectors in PER.C6 cells is attributed to defective Ad sequences in the pShuttleCMV vector since pAdApt-based vectors can generate high-titer and RCA-free Ad vectors in this cell line. The Ad sequences that may contribute to incompatibility between AdEasy and PER.C6 are identified in Ad nucleotides 342-454 and 3511-3533, as these two segments are present in pAdApt (sequence provide by Crucell) but missing in pShuttleCMV. Ad nucleotide numbering conforms to that of Chroboczek's numbering system (Chroboczek et al., 1992). The pIX promoter (Babiss and Vales, 1991) is intact in the pAdApt but defective in pShuttleCMV. pIX, as a capsid cement, participates in the stability of Ad particles (Rosa-Calatrava et al., 2001). There may also be other functions encoded by the Ad sequences that are missing in pShuttleCMV. The pShuttleCMV vector can be repaired by replacing the presumably defective sequence with its counterpart in pAdApt through homologous recombination in E. coli BJ5183 cells.

### Example 2

### Construction of pAdHighα

Crucell's shuttle plasmid pAdApt was separately digested with restriction enzymes SgrAI+EcoRI, and BstXI+EcoRI. In parallel, the shuttle plasmid pShuttleCMV was digested with SgrAI+BstXI. The resulting pAdApt SgrAI-EcoRI and BstXI-EcoRI fragments were inserted into the SgrAI-BstXI site of pShuttleCMV by 3-way ligation, resulting in a replication defective Ad vector. The replication-defective Ad vector encoding the influenza HA gene (Ad_{Highα}PNM2007/99.H3) was generated by transfecting the recombinant plasmid into PER.C6 cells. Cytopathic effects (CPE) emerged approximately 7 days after transfection, within the same timeframe as that required for the AdEasy system in 293 cells (He et al., 1998).

### Example 3

### Construction of pAdHighβ

To repair the defective sequences, pShuttleCMV's CMV promoter, the adjacent multiple cloning site, and flanking Ad sequences were replaced as one unit with their counterpart from pAdApt through homologous recombination, because these two shuttle plasmids share extensive overlapping sequences. However, a new marker was also required for selecting the recombinants. The full-length tetracycline (Tc) resistance gene (Backman and Boyer, 1983; Peden, 1983) from the plasmid pBR322 were amplified by PCR using primers 5′-GAGCTCGGTACCTTCTCATGTTTGACAGCTTATCAT-3′ and 5′-TCTAGAGGTACCAACGCTGCCCGAGATGCGCCGCGT-3′ with built-in KpnI sites. The amplified Tc gene was inserted into the KpnI site of the Amp-resistant plasmid pAdApt to generate a new plasmid pAdApt-Tc, which can be selected by applying both Amp and Tc to the growth medium.

The Ad sequence in pShuttleCMV was replaced with its counterpart in pAdApt-Tc using the high-efficiency AdEasier recombination protocol (Zeng et al., 2001). Briefly, pShuttleCMV was transformed into E. coli BJ5183 cells, and kanamycin (kan) resistance selected transformants. Kan-resistant cells were immediately transformed with pAdApt-Tc, and recombinants were selected by applying both Kan and Tc to the culture medium. Only when its counterpart in pAdApt-Tc, through homologous recombination replaced the indicated Ad sequence in pShuttleCMV, could the recombinant confer both Kan and Tc resistance to E. coli BJ5183 cells. The resultant pAdHighβ plasmid was purified from E. coli BJ5183 cells, transformed into E. coli DH10B cells as described (Zeng et al., 2001). The plasmid was subsequently validated by DNA sequencing.

### Example 4

### Construction of Adenovirus Vectors Encoding Influenza HA Using the AdHigh System

The KpnI fragments containing the A/Panama/2007/99 HA genes in the AdPNM2007/99.H3 vector was inserted into the KpnI site of pAdHigh-Tc to replace the Tc gene. The resultant plasmid was allowed to recombine with the Ad backbone plasmid pAdEasy1 in E. coli BJ5183 cells as described (Zeng et al., 2001). An Ad vector encoding the HA gene was generated in PER.C6 cells after transfection of the recombinant plasmid. The level of RCA contamination was not detectable out of 3×10¹¹ particles.

### Example 5

### Comparison of AdApt-, AdEasy-, and AdHighα-Derived Adenovirus Vectors

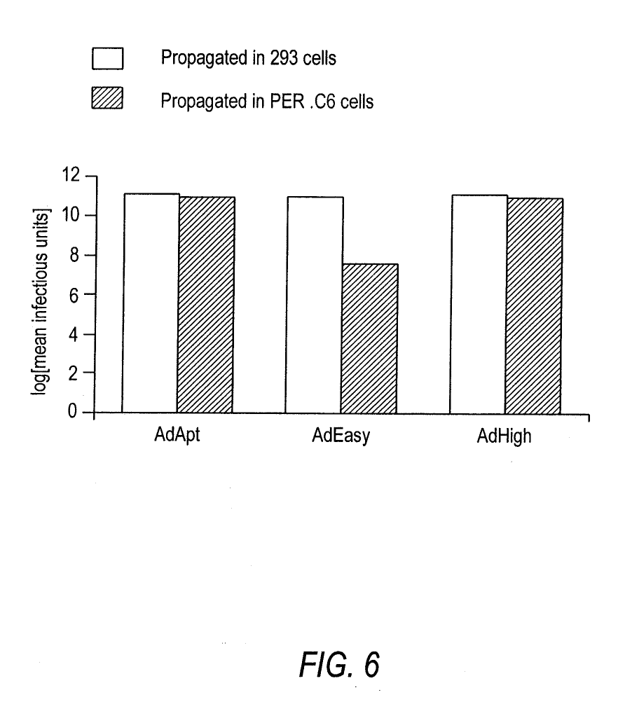

The propagation of AdApt-, AdEasy-, and AdHighα-derived adenovirus vectors encoding an influenza HA gene in 293 and PER.C6 cells was determined. Approximately 10⁶ cells were infected by Adendovirus vectors developed using one of AdApt, AdEasy, and AdHighα at an ifu-to-cell ratio of 25:1. Post-infection, cells were frozen for 2 days. After thawing, lysates were analyzed by the Adeno-X titer kit, as shown in FIG. 6. The data represent mean titers produced in a single well. Adenovirus vectors produced by AdApt and AdHighα exhibited no significant difference in the mean infectious units regardless of whether the vectors were propagated in PER.C6 cells or in 293 cells. In contrast, vectors produced by AdEasy resulted in a significant difference in the mean infectious units, with vectors propagated in 293 cells averaging an approximately 3-log decrease in mean infectious units when compared to counterparts propagated in PER.C6 cells.

The effectiveness of AdHighα-derived adenovirus vectors in eliciting hemagglutination-inhibition antibody titers was compared to that of AdApt-derived adenovirus vectors. ICR mice were immunized through intranasal administration with 2.5×10⁸ ifu of Ad_{H}PNM2007/99.H3 (AdHighα-derived) or AdPNM2007/99.H3 (AdApt-derived) vectors, each of which encoded the same influenza HA protein. One month post-immunization, sera was collected for hemagglutination-inhibition assay.

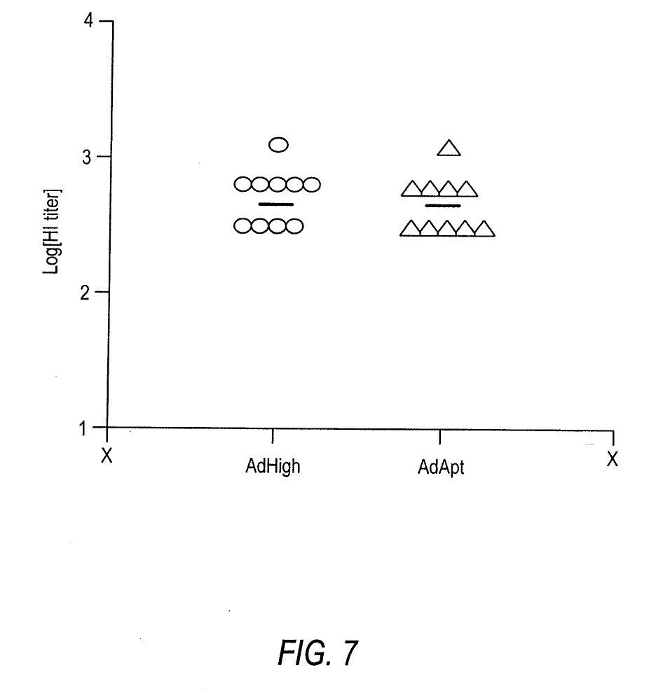

As seen in FIG. 7, nearly identical HI titers were obtained with both vectors, demonstrating that effectiveness of the adenovirus vector is not decreased through the use of the AdHighα-derived adenovirus vectors in comparison the AdApt-derived adenovirus vectors.

Having thus described in detail preferred embodiments of the present invention, it is to be understood that the invention defined by the appended claims is not to be limited by particular details set forth in the above description as many apparent variations thereof are possible without departing from the spirit or scope thereof.

### REFERENCES

1. Altschul, S. F., Gish, W., Miller, W., Myers, E. W., and Lipman, D. J. (1990) Basic local alignment search tool. J Mol. Biol. 215, 403-10.
2. Babiss, L. E., and Vales, L. D. (1991). Promoter of the adenovirus polypeptide IX gene: similarity to E1B and inactivation by substitution of the simian virus 40 TATA element. J Virol 65, 598-605.
3. Backman, K., and Boyer, H. W. (1983). Tetracycline resistance determined by pBR322 is mediated by one polypeptide. Gene 26, 197-203.
4. Baserga, R., and Denhardt, D. T. (eds.) (1992), Antisense Strategies, Annals of the New York Academy of Sciences. Vol. 600, New York Academy of Sciences, New York, N.Y.
5. Behr, J. P. (1994) Gene transfer with synthetic cationic amphiphiles: prospects for gene therapy. Bioconjug Chem. 5, 382-9.
6. Berk, A. J. (1986) Adenovirus promoters and E1A transactivation. Annu Rev Genet. 20, 45-79.
7. Bovamick, M. R., Miller, J. C., and Snyder, J. C. (1950) The influence of certain salts, amino acids, sugars, and proteins on the stability of rickettsiae. J. Bacteriol. 59, 509-22.
8. Brody, S. L., and Crystal, R. G. (1994) Adenovirus-mediated in vivo gene transfer. Ann N Y Acad. Sci. 716, 90-101; discussion 101-3.
9. Chartier, C., Degryse, E., Gantzer, M., Dieterle, A., Pavirani, A., and Mehtali, M. (1996). Efficient generation of recombinant adenovirus vectors by homologous recombination in Escherichia coli. J Virol 70, 4805-4810.
10. Chroboczek, J., Bieber, F., and Jacrot, B. (1992). The sequence of the genome of adenovirus type 5 and its comparison with the genome of adenovirus type 2. Virology 186, 280-285.
11. de la Luna, S., Fortes, P., Beloso, A., and Ortin, J. (1995) Influenza virus NS1 protein enhances the rate of translation initiation of viral mRNAs. J. Virol. 69, 2427-33.
12. Eckstein, F. (eds.) (1992) Oligonucleotides and Analogues, A Practical Approach, Oxford University Press, New York, N.Y.
13. Enami, K., Sato, T. A., Nakada, S., and Enami, M. (1994) Influenza virus NS1 protein stimulates translation of the M1 protein. J. Virol. 68, 1432-7.
14. Fallaux, F. J., Bout, A., van der Velde, I., van den Wollenberg, D. J., Hehir, K. M., Keegan, J., Auger, C., Cramer, S. J., van Ormondt, H., van der Eb, A. J., et al. (1998). New helper cells and matched early region 1-deleted adenovirus vectors prevent generation of replication-competent adenoviruses. Hum Gene Ther 9, 1909-1917.
15. Fields, B. N., Howley, P. M., Griffin, D. E., Lamb, R. A., Martin, M. A., Roizman, B., Straus, S. E., and Knipe, D. M. (eds) (2001) Fields—Virology, Lippincott, Williams, and Wilkins, Philadelphia, Pa.
16. Fortes, P., Beloso, A., and Ortin, J. (1994) Influenza virus NS1 protein inhibits pre-mRNA splicing and blocks mRNA nucleocytoplasmic transport. EMBO J. 13, 704-12.
17. Gorman, L., Suter, D., Emerick, V., Schumperli, D., and Kole, R. (1998) Stable alteration of pre-mRNA splicing patterns by modified U7 small nuclear RNAs. Proc Natl Acad Sci USA. 95, 4929-34.
18. Graham, F. L., and Prevec, L. (1995). Methods for construction of adenovirus vectors. Mol Biotechnol 3, 207-220.
19. Guo, Z. S., Wang, L. H., Eisensmith, R. C., and Woo, S. L. (1996) Evaluation of promoter strength for hepatic gene expression in vivo following adenovirus-mediated gene transfer. Gene Ther. 3, 802-10.
20. Havenga, M. J., Lemckert, A. A., Grimbergen, J. M., Vogels, R., Huisman, L. G., Valerio, D., Bout, A., and Quax, P. H. (2001) Improved adenovirus vectors for infection of cardiovascular tissues. J. Virol. 75, 3335-42.
21. He, T. C., Zhou, S., da Costa, L. T., Yu, J., Kinzler, K. W., and Vogelstein, B. (1998). A simplified system for generating recombinant adenoviruses. Proc Natl Acad Sci USA 95, 2509-2514.
22. Hilleman, M. R. (2002). Realities and enigmas of human viral influenza: pathogenesis, epidemiology and control. Vaccine 20, 3068-3087.
23. Hoffmann, E., Krauss, S., Pérez, D., Webby, R., and Webster, R. G. (2002). Eight-plasmid system for rapid generation of influenza virus vaccines. Vaccine 20, 3165-3170.
24. Jochemsen, A. G., Peltenburg, L. T., te Pas, M. F., de Wit, C. M., Bos, J. L., and van der Eb, A. J. (1987) Activation of adenovirus 5 E1A transcription by region E1B in transformed primary rat cells. EMBO J. 6, 3399-405.
25. Kozak, M. (1986). Point mutations define a sequence flanking the AUG initiator codon that modulates translation by eukaryotic ribosomes. Cell 44, 283-292.
26. Krug, R. M. and Soeiro, R. (1975) Studies on the intranuclear localization of influenza virus-specific proteins. Virology 64, 378-87.
27. Lu, Y., Qian, X. Y., and Krug, R. M. (1994) The influenza virus NS1 protein: a novel inhibitor of pre-mRNA splicing. Genes Dev. 8, 1817-28.
28. Marwick, C. (2000). Merits, flaws of live virus flu vaccine debated. JAMA 283, 1814-1815.
29. Mata, J. E., Joshi, S. S., Palen, B., Pirruccello, S. J., Jackson, J. D., Elias, N., Page, T. J., Medlin, K. L., and Iversen, P. L. (1997) A hexameric phosphorothioate oligonucleotide telomerase inhibitor arrests growth of Burkitt's lymphoma cells in vitro and in vivo. Toxicol Appl Pharmacol. 144, 189-97.
30. McEwan, N. R., and Gatherer, D. (1998) Adaptation of standard spreadsheet software for the analysis of DNA sequences. Biotechniques 24, 131-6, 138.
31. Milligan, J. F., Matteucci, M. D., and Martin, J. C. (1993) Current concepts in antisense drug design. J Med. Chem. 36, 1923-37.
32. Mills, C. K., and Gherna, R. L. (1988) Cryopreservation studies of Campylobacter. Cryobiology 25, 148-52.
33. Murakami, P., Pungor, E., Files, J., Do, L., van Rijnsoever, R., Vogels, R., Bout, A., and McCaman, M. (2002). A single short stretch of homology between adenoviral vector and packaging cell line can give rise to cytopathic effect-inducing, helper-dependent E1-positive particles. Hum Gene Ther 13, 909-920.
34. Nakamura, Y., Wada, K., Wada, Y., Doi, H., Kanaya, S., Gojobori, T., and Ikemura, T. (1996) Codon usage tabulated from the international DNA sequence databases. Nucleic Acids Res. 24, 214-5.
35. Neumann, G., Watanabe, T., Ito, H., Watanabe, S., Goto, H., Gao, P., Hughes, M., Perez, D. R., Donis, R., Hoffmann, E., et al (1999). Generation of influenza A viruses entirely from cloned cDNAs. Proc Natl Acad Sci USA 96, 9345-9350.
36. Nichol, K. L., Lind, A., Margolis, K. L., Murdoch, M., McFadden, R., Hauge, M., Magnan, S., and Drake, M. (1995). The effectiveness of vaccination against influenza in healthy, working adults. N Engl J Med 333, 889-893.
37. Osterhaus, A. D. and de Vries P. (1992) Vaccination against acute respiratory virus infections and measles in man. Immunobiology 184, 180-92.
38. Park, Y. W. and Katze, M. G. (1995) Translational control by influenza virus. Identification of cis-acting sequences and trans-acting factors which may regulate selective viral mRNA translation. J Biol. Chem. 270, 28433-9.
39. Peden, K. W. (1983). Revised sequence of the tetracycline-resistance gene of pBR322. Gene 22, 277-280.
40. Pfleiderer, M., Lower, J., and Kurth, R. (2001). Cold-attenuated live influenza vaccines, a risk-benefit assessment. Vaccine 20, 886-894.
41. Qiu, Y., Krug, R. M. (1994) The influenza virus NS1 protein is a poly(A)-binding protein that inhibits nuclear export of mRNAs containing poly(A). J. Virol. 68, 2425-32.
42. Qiu, Y., Nemeroff, M., and Krug, R. M. (1995) The influenza virus NS1 protein binds to a specific region in human U6 snRNA and inhibits U6-U2 and U6-U4 snRNA interactions during splicing. RNA 1, 304-16.
43. Regelson, W., Kuhar, S., Tunis, M., Fields, J., Johnson, J., Gluesenkamp, E. (1960) Synthetic polyelectrolytes as tumour inhibitors. Nature. 186, 778-80.
44. Remington, J. P. (1985) REMINGTON'S PHARMACEUTICAL SCIENCE, 17^{th} Edition, Mack Publishing Company, Easton, Pa., USA.
45. Robert, J. J., Gauffeny, I., Maccario, J., Jullien, C., Benoit, P., Vigne, E., Crouzet, J., Perricaudet, M., and Yeh, P. (2001). Degenerated pIX-IVa2 adenoviral vector sequences lowers reacquisition of the E1 genes during virus amplification in 293 cells. Gene Ther 8, 1713-1720.
46. Roberts, B. E., Miller, J. S., Kimelman, D., Cepko, C. L., Lemischka, I. R., and Mulligan, R. C. (1985) Individual adenovirus type 5 early region 1A gene products elicit distinct alterations of cellular morphology and gene expression. J. Virol. 56, 404-13.
47. Rosa-Calatrava, M., Grave, L., Puvion-Dutilleul, F., Chatton, B., and Kedinger, C. (2001). Functional analysis of adenovirus protein IX identifies domains involved in capsid stability, transcriptional activity, and nuclear reorganization. J Virol 75, 7131-7141.
48. Sambrook, J., Russell, D. W., and Sambrook, J. (2001) Molecular Cloning, Cold Spring Harbor Press, Cold Spring Harbor, N.Y.
49. Samstag, W., Eisenhardt, S., Offensperger, W. B., and Engels, J. W. (1996) Synthesis and properties of new antisense oligodeoxynucleotides containing benzylphosphonate linkages. Antisense Nucleic Acid Drug Dev. 6, 153-6.
50. Schmid, S. I., and Hearing, P. (1995) Selective encapsidation of adenovirus DNA. Curr Top Microbiol Immunol. 199, 67-80.
51. Shi, Z., Curiel, D. T., and Tang, D. C. (1999). DNA-based non-invasive vaccination onto the skin. Vaccine 17, 2136-2141.
52. Shi, Z., Zeng, M., Yang, G., Siegel, F., Cain, L. J., Van Kampen, K. R., Elmets, C. A., and Tang, D.C. (2001). Protection against tetanus by needle-free inoculation of adenovirus-vectored nasal and epicutaneous vaccines. J Virol 75, 11474-11482.
53. Strauss-Soukup, J. K., Vaghefi, M. M., Hogrefe, R. I., Maher, L. J., 3rd. (1997) Effects of neutralization pattern and stereochemistry on DNA bending by methylphosphonate substitutions. Biochemistry. 36, 8692-8.
54. Subbarao, K., Klimov, A., Katz, J., Regnery, H., Lim, W., Hall, H., Perdue, M., Swayne, D., Bender, C., Huang, J., et al. (1998). Characterization of an avian influenza A (H5N1) virus isolated from a child with a fatal respiratory illness. Science 279, 393-396.
55. Telling, G. C., Perera, S., Szatkowski-Ozers, M., and Williams, J. (1994) Absence of an essential regulatory influence of the adenovirus E1B 19-kilodalton protein on viral growth and early gene expression in human diploid W138, HeLa, and A549 cells. J. Virol. 68, 541-7.
56. Tinsley, J. M., Potter, A. C., Phelps, S. R., Fisher, R., Trickett, J. I., and Davies, K. E. (1996) Amelioration of the dystrophic phenotype of mdx mice using a truncated utrophin transgene. Nature 384, 349-53.
57. Tooze, J. (1980) DNA Tumor Viruses (Part 2): Moelcular Biology of Tumor Viruses, Cold Spring Harbor Laboratory Press, Cold Spring Harbor, N.Y.
58. Tsvetkov, T., and Brankova, R. (1983) Viability of micrococci and lactobacilli upon freezing and freeze-drying in the presence of different cryoprotectants. Cryobiology 20, 318-23.
59. van Ginkel, F. W., McGhee, J. R., Liu, C., Simecka, J. W., Yamamoto, M., Frizzell, R. A., Sorscher, E. J., Kiyono, H., and Pascual, D. W. (1997) Adenoviral gene delivery elicits distinct pulmonary-associated T helper cell responses to the vector and to its transgene. J. Immunol. 159, 685-93.
60. Van Kampen, K. R., Shi, Z., Gao, P., Zhang, J., Foster, K. W., Chen, D. T., Marks, D., Elmets, C. A., and Tang, D.C. (2005). Safety and immunogenicity of adenovirus-vectored nasal and epicutaneous influenza vaccines in humans. Vaccine.
61. Vincent, N., Ragot, T., Gilgenkrantz, H., Couton, D., Chafey, P., Gregoire, A., Briand, P., Kaplan, J. C., Kahn, A., and Perricaudet, M. (1993) Long-term correction of mouse dystrophic degeneration by adenovirus-mediated transfer of a minidystrophin gene. Nat. Genet. 5, 130-4.
62. von der Thusen, J. H., Fekkes, M. L., Passier, R., van Zonneveld, A. J., Mainfroid, V., van Berkel, T. J., and Biessen, E. A. (2004) Adenoviral transfer of endothelial nitric oxide synthase attenuates lesion formation in a novel murine model of postangioplasty restenosis. Arterioscler Thromb Vasc Biol. 24, 357-62.
63. Wang, T. T., Cheng, W. C., and Lee, B. H. (1998) A simple program to calculate codon bias index. Mol. Biotechnol. 10, 103-6.
64. White, E., Denton, A., and Stillman, B. (1988) Role of the adenovirus E1B 19,000-dalton tumor antigen in regulating early gene expression. J. Virol. 62, 3445-54.
65. Wolff, E., Delisle, B., Corrieu, G., and Gibert, H. (1990) Freeze-drying of Streptococcus thermophilus: a comparison between the vacuum and the atmospheric method. Cryobiology 27, 569-75.
66. Xiang, Z. Q., Yang, Y., Wilson, J. M., and Ertl, H. C. J. (1996). A replication-defective human adenovirus recombinant serves as a highly efficacious vaccine carrier. Virology 219, 220-227.
67. Zeng, M., Smith, S. K., Siegel, F., Shi, Z., Van Kampen, K. R., Elmets, C. A., and Tang, D. C. (2001). AdEasy system made easier by selecting the viral backbone plasmid preceding homologous recombination. Biotechniques 31, 260-262.
68. Zhu, J., Grace, M., Casale, J., Chang, A. T., Musco, M. L., Bordens, R., Greenberg, R., Schaefer, E., and Indelicato, S. R. (1999). Characterization of replication-competent adenovirus isolates from large-scale production of a recombinant adenoviral vector. Hum Gene Ther 10, 113-121.

1. A recombinant adenoviral vector comprising a first adenoviral sequence comprising SEQ ID NO: 1, a promoter sequence, a MCS, a transcriptional terminator, a second adenoviral sequence comprising SEQ ID NO: 2, a third adenoviral sequence comprising SEQ ID NO: 4, a bacterial origin of replication, and an antibiotic resistance gene, wherein SEQ ID NO: 2 and SEQ ID NO: 4 comprise sequences that allow homologous recombination to occur in a prokaryotic cell between the recombinant adenoviral shuttle plasmid and an adenoviral backbone plasmid to generate a recombinant plasmid capable of producing RCA-free Ad vectors in packaging cells.

2. The recombinant adenoviral vector of claim 1, wherein the promoter is selected from the group consisting of a cytomegalovirus (CMV) major immediate-early promoter, a simian virus 40 (SV40) promoter, a β-actin promoter, an albumin promoter, an Elongation Factor 1-α (EF1-α) promoter, a PγK promoter, a MFG promoter, and a Rous sarcoma virus promoter.

3. The recombinant adenoviral vector of claim 1, wherein the transcriptional terminator is a eukaryotic polyadenylation signal including the SV40 polyadenylation signal.

4. The recombinant adenoviral vector of claim 1, wherein the bacterial origin of replication can be derived from the pBR322 origin of replication.

5. The recombinant adenoviral vector of claim 1, wherein the antibiotic resistance genes are selected from the group consisting of ampicillin resistance gene, kanamycin resistance gene, chloramphenicol resistance gene, tetracycline resistance gene, hygromycin resistance gene, bleomycin resistance gene, and zeocin resistance gene.

6. The recombinant adenoviral vector of claim 1, wherein the prokaryotic cell is E. coli.

7. The recombinant adenoviral vector of claim 6, wherein the E. coli is BJ5183.

8. A recombinant adenoviral vector of claim 1, wherein the vector is pAdHigh.

9. A recombinant adenoviral vector comprising a first adenoviral sequence comprising sequences 1-454 derived from adenovirus serotype 5, a promoter sequence, a polylinker, a transcriptional terminator, a second adenoviral sequence comprising sequences 3511 to 5796 derived from adenovirus serotype 5, a third adenoviral sequence comprising sequences 34931-35935, a bacterial origin of replication, and an antibiotic resistance gene, wherein the second and third adenoviral sequences comprise sequences that allow homologous recombination to occur in a prokaryotic cell between the recombinant adenoviral shuttle plasmid and an adenoviral backbone plasmid.

10. The recombinant adenoviral vector of claim 9, wherein the promoter is selected from the group consisting of a cytomegalovirus (CMV) major immediate-early promoter, a simian virus 40 (SV40) promoter, a β-actin promoter, an albumin promoter, an Elongation Factor 1-α (EF1-α) promoter, a PγK promoter, a MFG promoter, a herpes virus promoter, and a Rous sarcoma virus promoter.

11. The recombinant adenoviral vector of claim 9, wherein the transcriptional terminator is a eukaryotic polyadenylation signal including the SV40 polyadenylation signal.

12. The recombinant adenoviral vector of claim 9, wherein the bacterial origin of replication can be derived from the pBR322 origin of replication.

13. The recombinant adenoviral vector of claim 9, wherein the antibiotic resistance genes are selected from the group consisting of ampicillin resistance gene, kanamycin resistance gene, chloramphenicol resistance gene, tetracycline resistance gene, hygromycin resistance gene, bleomycin resistance gene, and zeocin resistance gene.

14. The recombinant adenoviral vector of claim 9, wherein the prokaryotic cell is E. coli.

15. The recombinant adenoviral vector of claim 14, wherein the E. coli is BJ5183.

16. A recombinant adenoviral vector of claim 9, wherein the vector is pAdHigh.

17. A method of generating a recombinant adenovirus that is substantially free of replication-competent adenovirus (RCA), comprising:
a. Co-transforming a first shuttle plasmid and a second shuttle plasmid into a prokaryotic cell, wherein the first shuttle plasmid comprises a first adenoviral sequence and a first antibiotic resistance gene; and wherein the second shuttle plasmid comprises a second adenoviral sequence that contains adenoviral sequences not present in the first shuttle plasmid, and a second antibiotic resistance gene that is different from the first antibiotic resistance gene, wherein the co-transformation allows homologous recombination to occur between the first and second shuttle plasmids and wherein prokaryotic transformants expressing both of the antibiotic resistance genes in the first and second shuttle plasmids comprise a first recombined adenoviral plasmid;
b. Recovering the first recombined adenoviral plasmid from the prokaryotic cell;
c. Co-transforming the first recombined adenoviral plasmid and an adenoviral backbone plasmid into another prokaryotic cell, wherein the prokaryotic transformants comprise a second recombined adenoviral plasmid;
d. Recovering the second recombined adenoviral plasmid from the prokaryotic cell;
e. Transfecting PER.C6 packaging cells with the second recombined adenoviral plasmid; and
f. Recovering the recombinant adenovirus from the PER.C6 cells, wherein the recombinant adenovirus is substantially free of RCA.

18. The method of claim 17, wherein the first shuttle plasmid is pShuttle-CMV.

19. The method of claim 17, wherein the second shuttle plasmid is pAdApt-Tc.

20. The method of claim 17, wherein the antibiotic resistance genes are selected from the group consisting of ampicillin resistance gene, kanamycin resistance gene, chloramphenicol resistance gene, tetracycline resistance gene, hygromycin resistance gene, bleomycin resistance gene, and zeocin resistance gene.

21. The method of claim 17, wherein the additional adenoviral sequences not present in the pShuttleCMV comprise adenoviral sequences 342 to 454 from adenovirus serotype 5, and adenoviral sequences 3511 to 3533 from adenovirus serotype 5.

22. The method of claim 17, wherein the adenoviral backbone plasmid is pAdEasy1.

23. The method of claim 17, wherein the prokaryotic cell is E. coli.

24. The method of claim 23, wherein the E. coli is BJ5183.

25. A recombinant adenoviral vector generated by the method of claim 17.

26. A recombinant adenovirus generated by the method of claim 17.

27. A method of generating a recombinant adenovirus that is substantially free of replication-competent adenovirus (RCA), comprising:
a. Digesting a first and second shuttle plasmid with one or more restriction endonucleases, wherein the first shuttle plasmid comprises a first adenoviral sequence and wherein the second shuttle plasmid comprises additional adenoviral sequences not present in the first shuttle plasmid;
b. Excising a fragment encompassing the additional adenoviral sequences from the second shuttle plasmid;
c. Ligating the fragment containing additional adenoviral sequences into the first shuttle plasmid to replace the counterpart fragment, thereby resulting in a first recombined adenoviral plasmid;
d. Co-transforming the first recombined adenoviral plasmid and an adenoviral backbone plasmid into another prokaryotic cell, wherein the prokaryotic transformants comprise a second recombined adenoviral plasmid;
e. Recovering the second recombined adenovirus plasmid from the prokaryotic cell;
f. Transfecting the second recombined adenoviral plasmid into PER.C6 packaging cells; and
g. Recovering the recombinant adenovirus from the cells, wherein the recombinant adenovirus is substantially free of RCA.

28. The method of claim 27, wherein the first shuttle plasmid is pShuttleCMV.

29. The method of claim 27, wherein the second shuttle plasmid is pAdApt.

30. The method of claim 27, wherein the antibiotic resistance genes are selected from the group consisting of ampicillin resistance gene, kanamycin resistance gene, chloramphenicol resistance gene, tetracycline resistance gene, hygromycin resistance gene, bleomycin resistance gene, and zeocin resistance gene.

31. The method of claim 27, wherein the additional adenoviral sequences not present in pShuttleCMV comprise adenoviral sequences 342 to 454 from adenovirus serotype 5, and adenoviral sequences 3511 to 3533 from adenovirus serotype 5.

32. The method of claim 27, wherein the adenoviral backbone plasmid is pAdEasy1.

33. The method of claim 27, wherein the prokaryotic cell is E. coli.

34. The method of claim 33, wherein the E. coli is BJ5183.

35. A recombinant adenoviral vector generated by the method of claim 27.

36. A recombinant adenovirus generated by the method of claim 27.

37. An immunogenic composition comprising a recombinant adenovirus that is substantially free of replication-competent adenovirus (RCA) expressing one or more heterologous nucleic acids of interest, in admixture with pharmaceutically acceptable excipients.

38. The composition of claim 37, wherein the adenovirus that is substantially free of RCA is adenovirus serotype 5 (Ad5).

39. The composition of claim 37, wherein the adenovirus that is substantially free of RCA is generated by the method of claim 17.

40. The composition of claim 37, wherein the adenovirus that is substantially free of RCA is generated by the method of claim 27.

41. The composition of claim 37, wherein the one or more heterologous nucleic acids of interest comprise an influenza gene derived from influenza strains comprising influenza A, influenza B, influenza C, circulating recombinant forms, hybrid forms, clinical isolates, and field isolates.

42. The composition of claim 41, wherein the influenza gene comprises influenza hemagglutinin gene, influenza matrix gene, influenza neuraminidase gene, and influenza nuclear protein gene.

43. The composition of claim 37, further comprising an adjuvant.

44. An immunogenic composition comprising a recombinant adenovirus that is substantially free of replication-competent adenovirus (RCA) expressing one or more influenza immunogens, in admixture with pharmaceutically acceptable excipients.

45. The composition of claim 44, wherein the adenovirus that is substantially free of RCA is adenovirus serotype 5 (Ad5).

46. The composition of claim 44, wherein the adenovirus that is substantially free of RCA is generated by the method of claim 17.

47. The composition of claim 44, wherein the adenovirus that is substantially free of RCA is generated by the method of claim 27.

48. The composition of claim 44, wherein the one or more influenza immunogens comprise influenza hemagglutinin, influenza matrix, influenza neuraminidase, and influenza nuclear protein.

49. The composition of claim 44, wherein the one or more influenza immunogens are derived from influenza strains comprising influenza A, influenza B, influenza C, circulating recombinant forms, hybrid forms, clinical isolates, and field isolates.

50. The composition of claim 44, further comprising an adjuvant.

51. A method of expressing one or more heterologous nucleic acids in a recombinant adenovirus that is substantially free of replication-competent adenovirus (RCA), comprising:
a. Digesting a recombinant adenoviral vector of claim 1, 9, 25, or 35 with one or more restriction endonucleases, thereby linearizing the adenoviral vector;
b. Ligating one or more heterologous nucleic acids into the adenoviral vector, wherein the one or more heterologous nucleic acids are operably linked to a promoter sequence;
c. Transfecting the adenoviral vector into a mammalian packaging cell; and
d. Recovering the recombinant adenovirus expressing the one or more heterologous nucleic acids from the mammalian packaging cell.

52. The method of claim 51, wherein the adenovirus is derived from adenovirus serotype 5 (Ad5).

53. The method of claim 51, wherein the one or more heterologous nucleic acids comprise an influenza gene.

54. The method of claim 51, wherein the promoter sequence is selected from the group consisting of a cytomegalovirus (CMV) major immediate-early promoter, a simian virus 40 (SV40) promoter, a β-actin promoter, an albumin promoter, an Elongation Factor 1-α (EF1-α) promoter, a PγK promoter, a MFG promoter, a herpes virus promoter, and a Rous sarcoma virus promoter.

55. The method of claim 53, wherein the influenza gene comprises influenza hemagglutinin gene, influenza matrix gene, influenza neuraminidase gene, and influenza nuclear protein gene.

56. The method of claim 53, wherein the influenza gene is derived from influenza strains comprising influenza A, influenza B, influenza C, circulating recombinant forms, hybrid forms, clinical isolates, and field isolates.

57. A method of eliciting an immunogenic response to influenza in a subject in need thereof, comprising administering an immunologically effective amount of the composition of claim 44 to the subject.

58. The method of claim 57, wherein the influenza immunogen comprises influenza hemagglutinin, influenza matrix, influenza neuraminidase, and influenza nuclear protein.

59. The method of claim 57, wherein the influenza immunogen is derived from influenza strains comprising influenza A, influenza B, influenza C, circulating recombinant forms, hybrid forms, clinical isolates, and field isolates.

60. The method of claim 57, further comprising an adjuvant.

61. A method of introducing and expressing one or more heterologous nucleic acids in a cell of interest, comprising contacting the cell with a recombinant adenovirus that is substantially free of replication-competent adenovirus (RCA), wherein the recombinant adenovirus expresses the one or more heterologous nucleic acids, and culturing the cell or maintaining the animal under conditions sufficient to express the one or more heterologous nucleic acids.

62. The method of claim 61, wherein the cell is a human cell.

63. The method of claim 61, wherein the adenovirus is derived from adenovirus serotype 5 (Ad5).

64. The method of claim 61, wherein the one or more heterologous nucleic acids comprise influenza genes.

65. The method of claim 61, wherein the influenza gene comprises influenza hemagglutinin gene, influenza matrix gene, influenza neuraminidase gene, and influenza nuclear protein gene.

66. The method of claim 61, wherein the influenza gene is derived from influenza strains comprising influenza A, influenza B, influenza C, circulating recombinant forms, hybrid forms, clinical isolates, and field isolates.

67. A kit comprising a recombinant adenoviral vector of claim 1, an adenoviral backbone plasmid, and E. coli BJ 5183 cells.

68. The kit of claim 67, wherein the recombinant adenoviral shuttle vector is pAdHigh or a derivative of it.

69. The kit of claim 67, wherein the adenoviral backbone plasmid is pAdEasy1.
